(19) United States
(12) Patent Application Publication
(10) Pub. No.: US 2016/0374545 A1
(43) Pub. Date: Dec. 29, 2016

(54) ENDOSCOPE SYSTEM

(71) Applicant: OLYMPUS CORPORATION, Tokyo (JP)

(72) Inventors: Tatsuya OBARA, Tokyo (JP); Kazuki HONDA, Tokyo (JP); Yasuhito KURA, Tokyo (JP)

(73) Assignee: OLYMPUS CORPORATION, Tokyo (JP)

(21) Appl. No.: 15/262,203

(22) Filed: Sep. 12, 2016

Related U.S. Application Data
(63) Continuation of application No. PCT/JP2015/059112, filed on Mar. 25, 2015.

(30) Foreign Application Priority Data
Mar. 31, 2014 (JP) 2014-073513

(51) Int. Cl. A61B 1/06; A61B 1/05; A61B 1/07; A61B 1/00

(52) U.S. Cl. CPC A61B 1/0623; A61B 1/0646; A61B 1/00009; A61B 1/00006; A61B 1/051; A61B 1/07; A61B 1/00045

(57) ABSTRACT

An endoscope system includes: illuminating windows configured to emit a first illuminating light to the front and a second illuminating light to the side inside the subject; an observation window configured to acquire an image of the subject from the front; an observation window configured to acquire an image of the subject from the side; an image generation portion configured to generate a first image based on an image of the subject from the front and a second image based on an image of the subject from the side; a control portion configured to compare brightnesses of the first image and the second image; a polarization filter configured to adjust the amount of at least one of the first illuminating light and the second illuminating light; and a drive portion configured to drive a polarization filter on the basis of a result of the brightness comparison by the control portion.

### CROSS REFERENCE TO RELATED APPLICATION

This application is a continuation application of PCT/JP2015/059112 filed on Mar. 25, 2015 and claims benefit of Japanese Application No. 2014-073513 filed in Japan on Mar. 31, 2014, the entire contents of which are incorporated herein by this reference.

### BACKGROUND OF THE INVENTION

1. Field of the Invention

The present invention relates to an endoscope system, and more particularly to an endoscope system that radiates illuminating light in at least two directions and acquires a subject image from the at least two directions.

2. Description of the Related Art

Endoscopes are widely used in the medical field and industrial field. An endoscope includes illumination means and observation means on a distal end side of an insertion portion, and can be inserted into a subject to observe and inspect the inside of the subject.

In recent years, endoscopes that can observe two or more directions have been proposed. For example, as disclosed in Japanese Patent No. 4782900, an endoscope has been proposed which, in addition to a front field of view that takes the side to the front of the insertion portion as an observation field of view, also has a lateral field of view that takes the lateral face side of the insertion portion as an observation field of view. By using this kind of endoscope, the person performing the inspection can simultaneously observe two directions, i.e. the front direction and the lateral direction.

### SUMMARY OF THE INVENTION

An endoscope system according to one aspect of the present invention includes: an insertion portion to be inserted inside a subject; an illuminating light emitting portion configured to emit a first illuminating light toward a front region inside the subject which includes a front direction of the insertion portion that is approximately parallel to a longitudinal direction of the insertion portion, and to emit a second illuminating light toward a lateral region in which at least one part is different from the front region inside the subject and which includes a lateral direction of the insertion portion that intersects with the longitudinal direction of the insertion portion; a first subject image acquisition portion which is provided in the insertion portion and is configured to acquire a first subject image from the front region; a second subject image acquisition portion which is provided in the insertion portion and is configured to acquire a second subject image from the lateral region; an image generation portion configured to generate a front observation image based on the first subject image and generate a lateral observation image based on the second subject image; a brightness comparison portion configured to compare a brightness of the front observation image and a brightness of the lateral observation image; a light amount adjustment portion configured to adjust an amount of the second illuminating light; and a drive portion configured to drive the light amount adjustment portion so that the lateral observation image becomes approximately a same brightness as the front observation image, based on a result of comparing the brightnesses obtained by the brightness comparison portion.

### BRIEF DESCRIPTION OF THE DRAWINGS

FIG. 1 is a configuration diagram illustrating the configuration of an endoscope system relating to a first embodiment of the present invention;

FIG. 2 is a cross-sectional view of a distal end portion 6a of an insertion portion 6 that relates to the first embodiment of the present invention;

FIG. 3 is a view illustrating an example of a display screen of an endoscopic image displayed on a display apparatus 5 that relates to the first embodiment of the present invention;

FIG. 4 is a schematic view illustrating the configuration of a light guide 34 that relates to the first embodiment of the present invention;

FIG. 5 is a view illustrating the configuration of a polarization filter 31a that relates to the first embodiment of the present invention;

FIG. 6 is a view illustrating the configuration of a polarization filter 31b that relates to the first embodiment of the present invention;

FIG. 7 is a view for describing a distribution state between light amounts incident on a first region 63 and a second region 64 of the light guide 34 in a case where a rotational angle θ of the polarization filter 31b with respect to the polarization filter 31a is 0 degrees, that relates to the first embodiment of the present invention;

FIG. 8 is a view for describing a distribution state between light amounts incident on the first region 63 and the second region 64 of the light guide 34 in a case where a rotational angle θ of the polarization filter 31b with respect to the polarization filter 31a is 90 degrees, that relates to the first embodiment of the present invention;

FIG. 9 is a graph relating to the first embodiment of the present invention that illustrates the relation between the rotational angle θ of the polarization filter 31b and light amounts VL incident on the first region 63 and the second region 64 of the light guide 34;

FIG. 10 is a view for describing the brightness of an endoscopic image in a case where a side face of the distal end portion 6a of the insertion portion 6 is close to an inner wall inside a subject, that relates to the first embodiment of the present invention;

FIG. 11 is a view for describing the brightness of an endoscopic image in a case where a distal end face of the distal end portion 6a of the insertion portion 6 is close to an inner wall inside a subject, that relates to the first embodiment of the present invention;

FIG. 12 is a configuration diagram that illustrates the configuration of an endoscope system that relates to a second embodiment of the present invention;

FIG. 13A is a view illustrating an example of a display screen of endoscopic images displayed on the display apparatus 5 that relates to the second embodiment of the present invention;

FIG. 13B is a view illustrating an example of display screens of endoscopic images displayed on a plurality of the display apparatuses 5 that relates to the second embodiment of the present invention;

FIG. 14 is a schematic view illustrating the configuration of a light guide 34A that relates to the second embodiment of the present invention;

FIG. 15 is a view illustrating the configuration of a polarization filter 81a that relates to the second embodiment of the present invention;

FIG. 16 is a view illustrating the configuration of a polarization filter 81b that relates to the second embodiment of the present invention;

FIG. 17 is a view illustrating a correspondence relation between three regions 91, 92 and 93 of a proximal end portion 34a of the light guide 34A and respective areas R3, R4, R5 and R6 of three polarization filters 31a, 81a and 81b that relates to the second embodiment of the present invention;

FIG. 18 is a graph illustrating the relation between a rotational angle θ1 of the polarization filter 81a with respect to the polarization filter 31a, and a light amount VL incident on the region 93 and light amount VL incident on the region 92 of the light guide 34, that relates to the second embodiment of the present invention;

FIG. 19 is a graph illustrating the relation between a rotational angle θ2 of the polarization filter 81b with respect to the polarization filter 81a, and a light amount incident on the region 92 and light amount incident on the region 91 of the light guide 34, that relates to the second embodiment of the present invention;

FIG. 20 is a view illustrating a correspondence relation between the three regions 91, 92 and 93 of the proximal end portion of the light guide 34A and three polarization filters 31a, 81a1 and 81b as a light amount adjustment portion that relates to a modification of the second embodiment of the present invention;

FIG. 21 is a graph illustrating the relation between a rotational angle θ3 of the polarization filter 81a1 with respect to the polarization filter 31a, and a light amount VL that is incident on the region 93 of the light guide 34A, that relates to the modification of the second embodiment of the present invention;

FIG. 22 is a graph illustrating the relation between a rotational angle θ4 of the polarization filter 81b with respect to a polarization filter 31a, and a light amount that is incident on the region 92 and a light amount that is incident on the region 91 of the light guide 34A, that relates to the modification of the second embodiment of the present invention;

FIG. 23 is a configuration diagram illustrating the configuration of an endoscope system relating to a third embodiment of the present invention;

FIG. 24 is a schematic view illustrating the configuration of a light guide 34B that relates to the third embodiment of the present invention;

FIG. 25 is a view relating to the third embodiment of the present invention which illustrates the configuration of a polarization filter 100 and also illustrates a correspondence relation between three regions 101, 102 and 103 of a proximal end portion of the light guide 34B and respective areas R0, R′ (R71, R72), R8 and R9 of the polarization filter 31a and polarization filter 100;

FIG. 26 is a graph relating to the third embodiment of the present invention which illustrates the relation between a rotational angle θ5 of the polarization filter 100 with respect to the polarization filter 31a, and light amounts incident on respective regions of the light guide 34B;

FIG. 27 is a view relating to a modification of the third embodiment of the present invention which illustrates a correspondence relation between three regions 104, 105 and 106 of an incident surface 62A of the light guide 34B and two polarization filters 31a and 100a as a light amount adjustment portion;

FIG. 28 is a configuration diagram illustrating the configuration of an endoscope system relating to a fourth embodiment of the present invention;

FIG. 29 is a view relating to the fourth embodiment of the present invention which illustrates the configuration of polarization filters 111 and 112 as a light amount adjustment portion, and also illustrates a correspondence relation between four regions 121, 122, 123 and 124 of a proximal end portion of a light guide 34C and respective areas R21, R22, R23 and R24 of the two polarization filters 111 and 112;

FIG. 30 is a chart relating to the fourth embodiment of the present invention which is used to describe a rotation angle θ7 and light amounts incident on an incident surface 62A of the light guide 34C;

FIG. 31 is a configuration diagram illustrating the configuration of an endoscope system relating to a fifth embodiment of the present invention;

FIG. 32 is a schematic view illustrating the configuration of a light guide 34D that relates to the fifth embodiment of the present invention;

FIG. 33 is a view relating to the fifth embodiment of the present invention which illustrates a correspondence relation between four regions 131, 132, 133 and 134 of the incident surface 62A of the light guide 34D and respective areas R31, R32, R33, R34 and R35 of the three polarization filters 31a, 121 and 122;

FIG. 34 is a view relating to the fifth embodiment of the present invention which illustrates an example of a display screen of an endoscopic image that is displayed on the display apparatus 5;

FIG. 35 is a graph relating to the fifth embodiment of the present invention which illustrates the relation between a rotational angle θ8 of a polarization filter 113 with respect to the polarization filter 31a, and a light amount VL incident on a region 134 and a light amount VL incident on three regions 131, 132 and 133 of the light guide 34D;

FIG. 36 is a graph relating to the fifth embodiment of the present invention which illustrates the relation between a rotational angle θ9 of a polarization filter 114 with respect to the polarization filter 113 and light amounts incident on respective regions 131, 132 and 133 of the light guide 3D;

FIG. 37 is a configuration diagram illustrating the configuration of an endoscope system relating to a sixth embodiment of the present invention; and

FIG. 38 is a view relating to the sixth embodiment of the present invention which illustrates a correspondence relation between four regions 131, 132, 133 and 134 of the proximal end portion of the light guide 34D and respective areas R41, R42, R43, R44, R45 and R46 of the four polarization filters 131, 132, 133 and 134.

### DETAILED DESCRIPTION OF THE PREFERRED EMBODIMENT(S)

Hereunder, embodiments of the present invention are described with reference to the drawings.

### First Embodiment

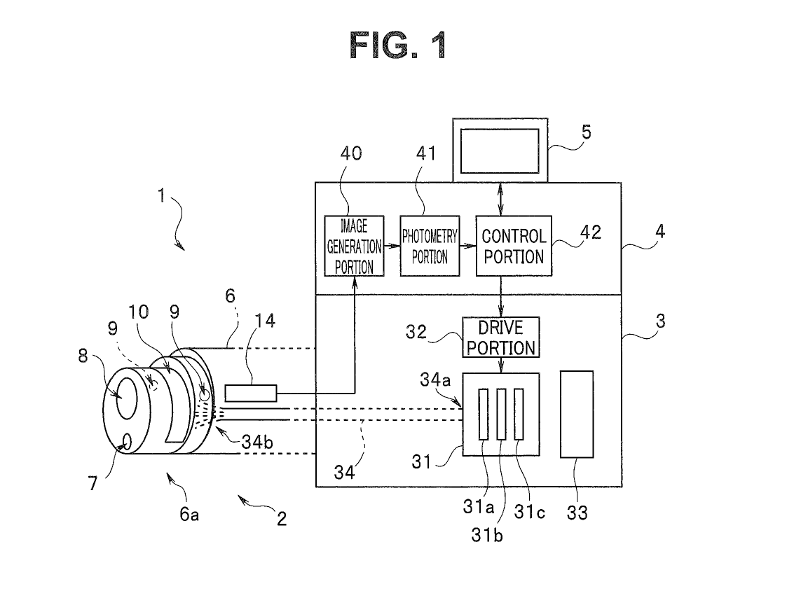

FIG. 1 is a configuration diagram that illustrates the configuration of an endoscope system relating to the present embodiment. An endoscope system 1 includes an endoscope 2, a light source apparatus 3, a processor 4 and a display apparatus 5.

The endoscope 2 includes an insertion portion 6 to be inserted into a subject, and an unshown operation portion, and is connected by unshown cables to the light source apparatus 3 and the processor 4. An illuminating window 7 and an observation window 8 for front observation, and two illuminating windows 9 and an observation window 10 for lateral observation are provided in a distal end portion 6a of the insertion portion 6 of the endoscope 2.

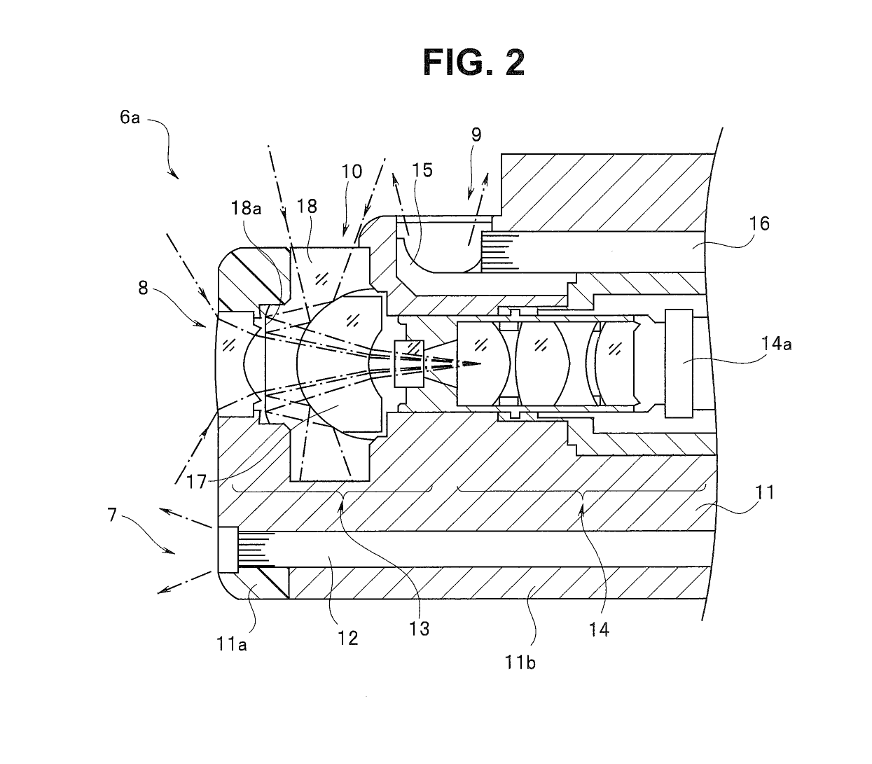

FIG. 2 is a cross-sectional view of the distal end portion 6a of the insertion portion 6. Note that, in FIG. 2, only one illuminating window 9 for lateral observation is shown.

The distal end portion 6a of the insertion portion 6 has a distal end rigid member 11, and the illuminating window 7 is provided in a distal end face of the distal end rigid member 11. A distal end face of a light guide for front illumination 12 is arranged at the rear side of the illuminating window 7. The observation window 8 is provided in the distal end face of the distal end rigid member 11. An objective optical system 13 is arranged on the rear side of the observation window 8. An image pickup unit 14 is arranged on the rear side of the objective optical system 13. Note that a cover 11a is attached to the distal end portion of the distal end rigid member 11. Further, the insertion portion 6 is covered with an outer covering 11b.

Hence, illuminating light for the front is emitted from the illuminating window 7, and reflected light from the subject as an observation site inside the subject is incident on the observation window 8.

Two illuminating windows 9 are arranged in a side face of the distal end rigid member 11. A distal end face of a light guide for lateral illumination 16 is arranged to the rear of each illuminating window 9 through a mirror 15 whose reflective surface is a curved surface.

Hence, the illuminating window 7 and the plurality of illuminating windows 9 constitute an illuminating light emitting portion configured to emit, inside the subject, a first illuminating light in the front direction as a first direction and a second illuminating light in the lateral direction as a second direction that includes a direction that is different to the first direction.

The observation window 10 is arranged in the side face of the distal end rigid member 11. The objective optical system 13 is arranged on the rear side of the observation window 10. The objective optical system 13 is configured so as to direct reflected light from the front that passed through the observation window 8 and reflected light from the side that passed through the observation window 10 to the image pickup unit 14. In FIG. 2, the objective optical system 13 has two optical members 17 and 18. The optical member 17 is a lens that has a convex surface 17a. The optical member 18 has a reflective surface 18a that reflects light from the convex surface 17a of the optical member 17 towards the image pickup unit 14 through the optical member 17.

That is, the observation window 8 constitutes a subject image acquisition portion that is provided in the insertion portion 6 and is configured to acquire an image from the front as a first direction, and the observation window 10 constitutes a subject image acquisition portion that is provided in the insertion portion 6 and is configured to acquire an image from the side as a second direction. The observation window 10 is disposed to the proximal end side of the insertion portion 6 with respect to the observation window 8.

More specifically, an image from the front as the first direction is a subject image from a front-view direction (first direction) that includes the front of the insertion portion 6 that is approximately parallel to the longitudinal direction of the insertion portion 6, that is, a subject image of a first region, and an image from the side as a second direction is a subject image from a side-view direction (second direction) that includes the sides of the insertion portion 6 that is a direction that intersects with the longitudinal direction of the insertion portion 6, that is, a subject image of a second region of the subject. Further, the observation window 8 is a front subject image acquisition portion configured to acquire a subject image of a first region inside the subject that includes the front of the insertion portion 6, and the observation window 10 is a lateral subject image acquisition portion configured to acquire a subject image of a second region inside the subject that includes the sides of the insertion portion 6.

The observation window 8 that is a subject image acquisition portion is disposed facing the direction in which the insertion portion 6 is to be inserted in the distal end portion 6a of the insertion portion 6. The observation window 10 that is a subject image acquisition portion is disposed facing an outer diameter direction of the insertion portion 6 in the distal end portion 6a of the insertion portion 6. The image pickup unit 14 that is an image pickup portion is disposed so as to photoelectrically convert a subject image from the observation window 8 and a subject image from the observation window 10 with the same image pickup surface, and is electrically connected to an image generation portion 40 of the processor 4 that is an image processing portion.

Hence, illuminating light for the front is emitted from the illuminating window 7 and reflected light from the subject passes through the observation window 8 and is incident on the image pickup unit 14, and illuminating light for the sides is emitted from the two illuminating windows 9 and reflected light from the subject passes through the observation window 10 and is incident on the image pickup unit 14. An image pickup device 14a of the image pickup unit 14 photoelectrically converts an optical image of the subject, and outputs an image pickup signal to the processor 4.

Returning to FIG. 1, the image pickup signal from the image pickup unit 14 is supplied to the processor 4 that is an image processing portion, and an endoscopic image is generated by a processing circuit such as the image generation portion 40. The endoscopic image is outputted to the display apparatus 5 from the processor 4. The image generation portion 40 generates a first image based on a first subject image and a second image based on a second subject image.

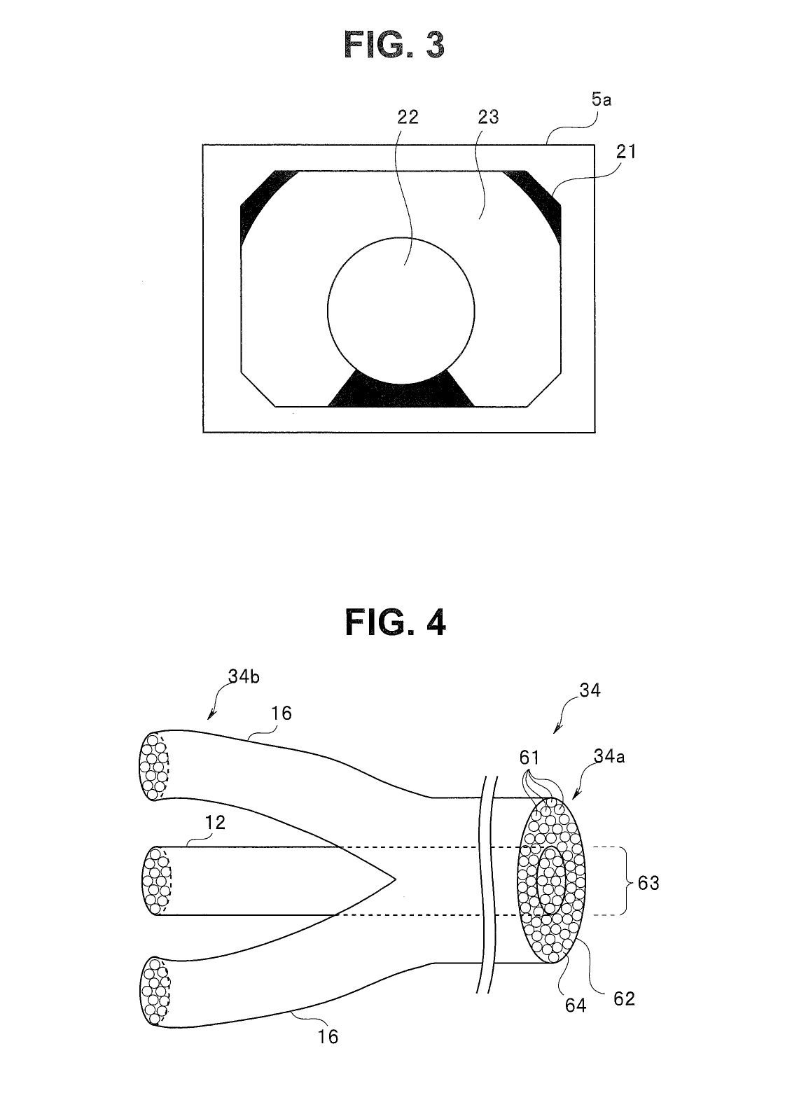

FIG. 3 is a view illustrating an example of a display screen of an endoscopic image that is displayed by the display apparatus 5.

An endoscopic image 21 displayed on a display screen 5a of the display apparatus 5 is an approximately rectangular image that has two regions 22 and 23. A circular region 22 at a center part is a region that displays a front observation image. The front observation image corresponds to a first image that corresponds to a subject image of a first region of the subject.

A C-shaped region 23 around the region 22 at the center part of the endoscopic image 21 is a region that displays a lateral observation image. The lateral observation image corresponds to a second image that corresponds to a subject image of a second region of the subject.

That is, the front observation image is displayed on the display screen 5a of the display apparatus 5 so as to be a substantially circular shape, and the lateral observation image is displayed on the display screen 5a of the display apparatus 5 so as to be an annular shape that surrounds at least part of the circumference of the front observation image. Hence, a wide-angle endoscopic image is displayed on the display apparatus 5.

Although this kind of image is realized by using a double reflection optical system that causes return light to be reflected twice with a side-view mirror lens, a configuration may also be adopted in which return light from a subject is reflected once by a single reflection optical system, and the reflected light is subjected to image processing by the processor 4 to align the orientations of a side-view field of view image and a direct-view field of view image.

Note that boundary regions of the first subject image and the second subject image may overlap or not overlap. In the case of a state in which the aforementioned boundary regions are overlapping, overlapping subject images may be acquired with the first subject image acquisition portion and the second subject image acquisition portion.

The light source apparatus 3 includes a light adjustment portion 31, a drive portion 32 that drives the light adjustment portion 31, and a light source 33.

The light adjustment portion 31 includes two polarization filters 31a and 31b and a diaphragm 31c. The configurations of the two polarization filters 31a and 31b are described later. The diaphragm 31c regulates an amount of light from the light source 33 based on a diaphragm control signal from a control portion 42.

In the light adjustment portion 31, the amount of light from the light source 33 is adjusted by the diaphragm 31c, and the two polarization filters 31a and 31b adjust the light amount so that the brightness of respective images displayed in the regions 22 and 23 becomes appropriate.

The two polarization filters 31a and 31b constitute a light amount adjustment portion configured to adjust a light amount of at least one of the first illuminating light that is emitted to the front and the second illuminating light that is emitted to the sides in order to adjust the brightness of the lateral observation image with respect to the front observation image. The plurality of polarization filters as an example of a light amount adjustment portion have, for example, respective portions that are disposed substantially collinearly so as to lie along the optical axis of the illuminating light.

Light emitted from the light adjustment portion 31 is condensed at a proximal end portion 34a of the light guide 34 by an unshown light condensing apparatus. The light emitted from the light adjustment portion 31 passes through the light guide 34 and is emitted from a distal end portion 34b of the light guide 34.

The light guide 34 includes the light guide for front illumination 12 and the light guide for lateral illumination 16 that are described above.

FIG. 4 is a schematic view that illustrates the configuration of the light guide 34. The light guide 34 is constituted by bundling a large number of optical fibers 61. The proximal end portion 34a of the light guide 34 has a circular incident surface 62 on which light that passes through the light adjustment portion 31 is incident. End faces of the large number of optical fibers 61 are gathered together at the incident surface 62.

The incident surface 62 has two regions 63 and 64 that are light-receiving regions. An optical fiber group having end portions in the first region 63 is the light guide for front illumination 12. An optical fiber group having end portions in the second region 64 is the light guide for lateral illumination 16.

That is, the incident surface 62 of the proximal end portion 34a of the light guide 34 constitutes a light-receiving portion configured to receive illuminating light for illuminating the inside of the subject that is supplied from the light source 33, at the region 63 and the region 64 which are at a central part and a peripheral part, respectively, in the cross-sectional direction of the light guide 34. Further, the light guide 34 constitutes a light-guiding portion configured to guide light into the insertion portion 6 and emit illuminating light that is received at the region 63 at the central part in a first direction that is the front direction, and emit illuminating light that is received at the region 64 at the peripheral part in a second direction that is the lateral direction.

In FIG. 4, proximal end portions of the light guide for front illumination 12 are held together and disposed in a circular shape in the region 63 at the center part of the circular incident surface 62 of the proximal end portion 34a of the light guide 34. Proximal end portions of the light guide for lateral illumination 16 are held together and disposed in the region 64 that is a circular ring-shaped portion around the circular region 63 of the proximal end portion 34a of the light guide 34.

Note that a partition film may be provided so that light does not leak between the light guide for front illumination 12 and the light guide for lateral illumination 16.

Returning to FIG. 1, the drive portion 32 is a drive circuit configured to drive the two polarization filters 31a and 31b so that at least one of the two polarization filters 31a and 31b is rotated. Although in the following description the polarization filter 31b is rotated, a configuration may also be adopted in which the polarization filter 31a is rotated. Hence, as described later, the drive portion 32 constitutes a drive portion configured to drive the polarization filter 31b that is a light amount adjustment portion, based on a result of comparing the brightnesses of a front observation image and a lateral observation image at the control portion 42.

The light source 33 has a lamp that emits white light.

The processor 4 includes a photometry portion 41 and the control portion 42. The photometry portion 41 is a processing portion configured to calculate the respective brightnesses of the two regions 22 and 23 of the endoscopic image 21 that is described above, based on image data for an endoscopic image that is generated in the processor 4. The photometry portion 41 calculates the brightness of the region 22 and the brightness of the region 23, and outputs the calculated values to the control portion 42. The brightness of the respective regions is the average value of the luminance of all pixels inside the respective regions.

The respective configurations of the two polarization filters 31a and 31b of the light adjustment portion 31 will now be described.

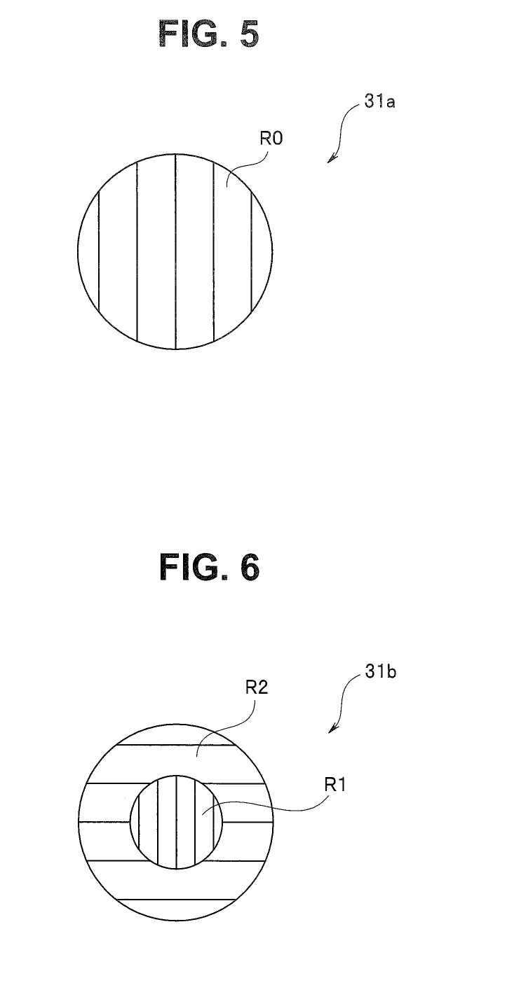

FIG. 5 is a view that illustrates the configuration of the polarization filter 31a. The polarization filter 31a is a circular polarization filter in which slits are provided in the lengthwise direction in an entire area R0, and is fixed so as not to rotate with respect to the proximal end portion 34a of the light guide 34. The plurality of lengthwise slits are provided side by side at regular intervals. In the case illustrated in FIG. 5, when light is passing through the polarization filter 31a, only light which vibrates in the lengthwise direction passes therethrough.

FIG. 6 is a view that illustrates the configuration of the polarization filter 31b. The polarization filter 31b is a circular polarization filter in which lengthwise slits having the same width as the slits of the polarization filter 31a are provided in an area R1, and crosswise slits having the same width as the slits of the polarization filter 31a are provided in a circular ring-shaped area R2, and which is disposed so as to be rotatable around the central axis of the circle. That is, the direction of the slits in the area R1 and the direction of the slits in the area R2 are orthogonal.

The area R1 is circular, and the area R2 is a circular ring-shaped region around the area R1. In the case illustrated in FIG. 6, light that passes through the area R1 is light which vibrates in the lengthwise direction, and light that passes through the area R2 is light which vibrates in the crosswise direction.

Rotating of the polarization filter 31b is performed by the drive portion 32 under control of the control portion 42.

The polarization filter 31a and polarization filter 31b are disposed on the same axis as the diaphragm 31c. The amount of light from the light source 33 is adjusted by the diaphragm 31c. Light that passed through the diaphragm 31c is transmitted through the polarization filter 31b and is incident on the polarization filter 31a, and light transmitted through the polarization filter 31a is incident on the incident surface 62 of the proximal end portion 34a of the light guide 34.

The light adjustment portion 31 is disposed with respect to the light guide 34 so that light emitted from the area R1 is transmitted through the polarization filter 31a and is incident on the first region 63 of the incident surface 62, and light emitted from the area R2 is transmitted through the polarization filter 31a and is incident on the second region 64 of the incident surface 62.

In this case, the outer diameters of the polarization filters 31a and 31b and the outer diameter of the incident surface 62 of the light guide 34 are equal, and the outer diameter of the area R1 of the polarization filter 31b and the outer diameter of the first region 63 of the incident surface 62 are equal.

The distribution ratio between the light amount that passes through the area R1 and is incident on the first region 63 of the light guide 34 and the light amount that passes through the area R2 and is incident on the second region 64 of the light guide 34 can be changed by rotating the polarization filter 31b within a range from 0 degrees to 90 degrees. That is, the two light amounts of the illumination for a front observation image and the illumination for a lateral observation image can be balanced by rotating the polarization filter 31b within a range from 0 degrees to 90 degrees relative to the polarization filter 31a. Further, the overall amount of illuminating light can be controlled by controlling the diaphragm 31c.

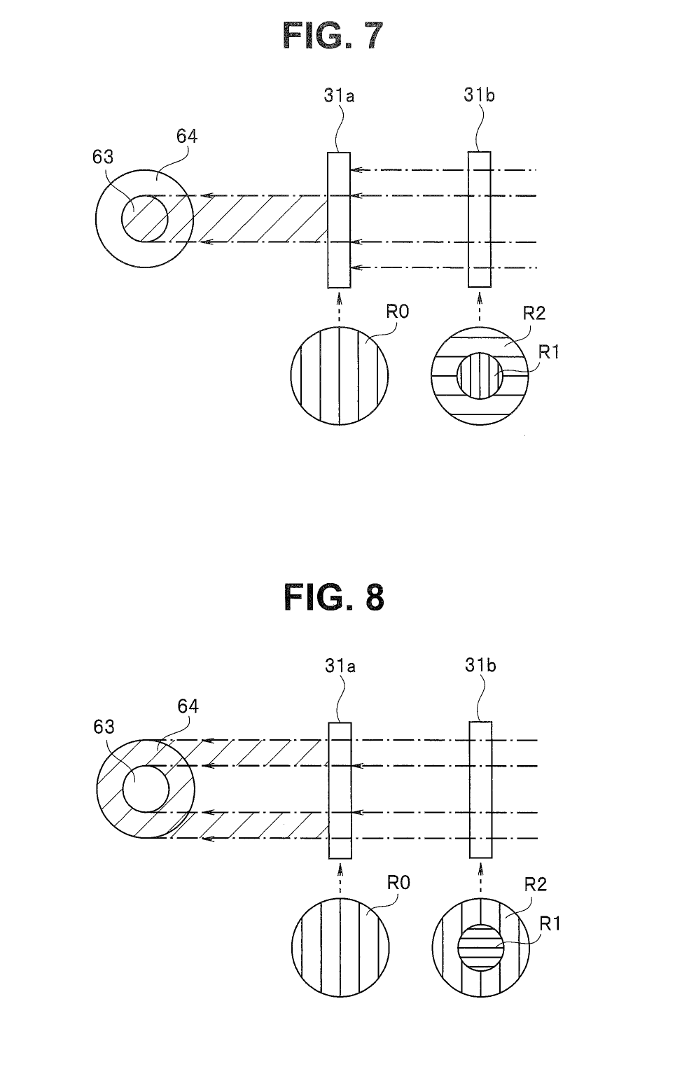

FIG. 7 and FIG. 8 are views for describing changes in the distribution of the light amount incident on the first region 63 and the light amount incident on the second region 64 of the light guide 34.

FIG. 7 is a view for describing the distribution state between the light amounts incident on the first region 63 and the second region 64 of the light guide 34 in a case where a rotational angle θ of the polarization filter 31b relative to the polarization filter 31a is 0 degrees.

In this case, the rotational angle θ of the polarization filter 31b relative to the polarization filter 31a when the direction of the slits in the polarization filter 31a and the direction of the slits in the area R1 of the polarization filter 31b are parallel is taken as 0 degrees.

When the rotational angle is 0 degrees, as indicated by diagonal lines in FIG. 7, although 100% of the light that has been transmitted through the area R1 is transmitted though the polarization filter 31a, the light that has been transmitted through the area R2 cannot pass through the polarization filter 31a. This is because the direction of the slits of the area R2 is orthogonal to the direction of the slits of the polarization filter 31a.

FIG. 8 is a view for describing the distribution state between the light amounts incident on the first region 63 and the second region 64 of the light guide 34 in a case where the rotational angle θ of the polarization filter 31b relative to the polarization filter 31a is 90 degrees.

In the case illustrated in FIG. 8, when the direction of the slits in the polarization filter 31a and the direction of the slits in the area R1 of the polarization filter 31b are orthogonal to each other, the rotational angle θ of the polarization filter 31b relative to the polarization filter 31a is 90 degrees.

When the rotational angle θ is 90 degrees, as indicated by diagonal lines in FIG. 8, although 100% of the light that has been transmitted through the area R2 is transmitted though the polarization filter 31a, the light that has been transmitted through the area R1 cannot pass through the polarization filter 31a. This is because the direction of the slits of the area R1 is orthogonal to the direction of the slits of the polarization filter 31a.

When the rotational angle θ of the polarization filter 31b is changed within the range from 0 to 90 degrees, the distribution state between the light amounts incident on the first region 63 and the second region 64 of the light guide 34 changes.

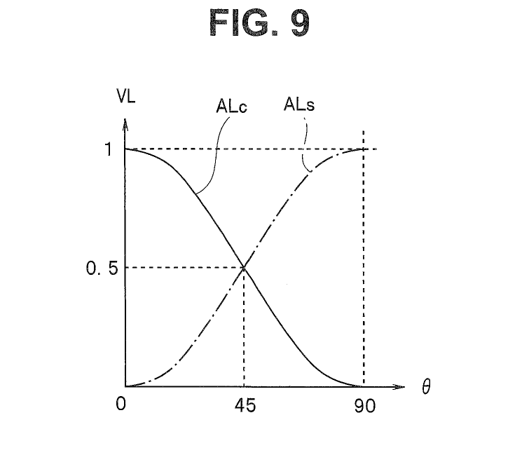

FIG. 9 is a graph illustrating the relation between the rotational angle θ of the polarization filter 31b and light amounts VL incident on the first region 63 and the second region 64 of the light guide 34.

When the rotational angle θ of the polarization filter 31b changes from 0 degrees toward 90 degrees, the light amount VL incident on the first region 63 of the light guide 34 gradually decreases as shown by a solid line ALc, and the light amount VL incident on the second region 64 of the light guide 34 gradually increases as shown by an alternate long and short dashed line ALs.

When the rotational angle θ of the polarization filter 31b is 0 degrees, as shown in FIG. 7, the light amount VL that is incident on the first region 63 is 1 (that is, 100% transmission), and the light amount VL that is incident on the second region 64 is 0 (that is, 0% transmission). When the rotational angle θ of the polarization filter 31b is 90 degrees, as shown in FIG. 8, the light amount VL that is incident on the first region 63 is 0 (that is, 0% transmission), and the light amount VL that is incident on the second region 64 is 1 (that is, 100% transmission).

When the rotational angle θ of the polarization filter 31b is 45 degrees, the light amount VL that is incident on the first region 63 and the light amount VL that is incident on the second region 64 are each 0.5 (that is, 50% transmission).

Thus, by changing the rotational angle θ of the polarization filter 31b within the range from 0 to 90 degrees, the distribution between the two light amounts VL that are incident on the first region 63 and the second region 64 of the light guide 34 can be changed.

Next, operations of the processor 4 are described.

As described above, the photometry portion 41 calculates the brightness of the region 22 that displays the front observation image and the brightness of the region 23 that displays the lateral observation image in an endoscopic image, and outputs the calculated brightness values to the control portion 42. The control portion 42 compares a brightness La of the region 22 that displays the front observation image and a brightness Lb of the region 23 that displays the lateral observation image, and drives the drive portion 32 to rotate the polarization filter 31b so that the brightness La and the brightness Lb become equal. The overall brightness of the endoscopic image 21 is adjusted by the control portion 42 controlling the diaphragm 31c. That is, the control portion 42 controls the drive portion 32 so that the drive portion 32 drives the light adjustment portion 31 so that the brightnesses of the two images as photometry results from the photometry portion 41 become the same.

Hence, the control portion 42 constitutes a brightness comparison portion configured to compare the brightness of a first image that is a front observation image and a second image that is a lateral observation image.

The control portion 42, for example, compares the brightnesses La and Lb while monitoring the brightnesses, and when the brightness La is greater than the brightness Lb, drives the drive portion 32 in a range in which the rotational angle θ is from 45 degrees to 90 degrees, and when the brightness La is less than the brightness Lb, drives the drive portion 32 in a range in which the rotational angle θ is from 0 degrees to 45 degrees, so as to thus make the brightnesses La and Lb become equal. That is, rotational control of the polarization filter 31b is performed by feedback control so that the brightnesses La and Lb become equal.

In addition, even when the brightnesses La and Lb are equal, when the brightnesses are not a predetermined appropriate brightness L0, the brightness of the region 22 and the brightness of the region 23 in the endoscopic image can be controlled to become equal and the endoscopic image can be controlled to an appropriate brightness by controlling the diaphragm 31c.

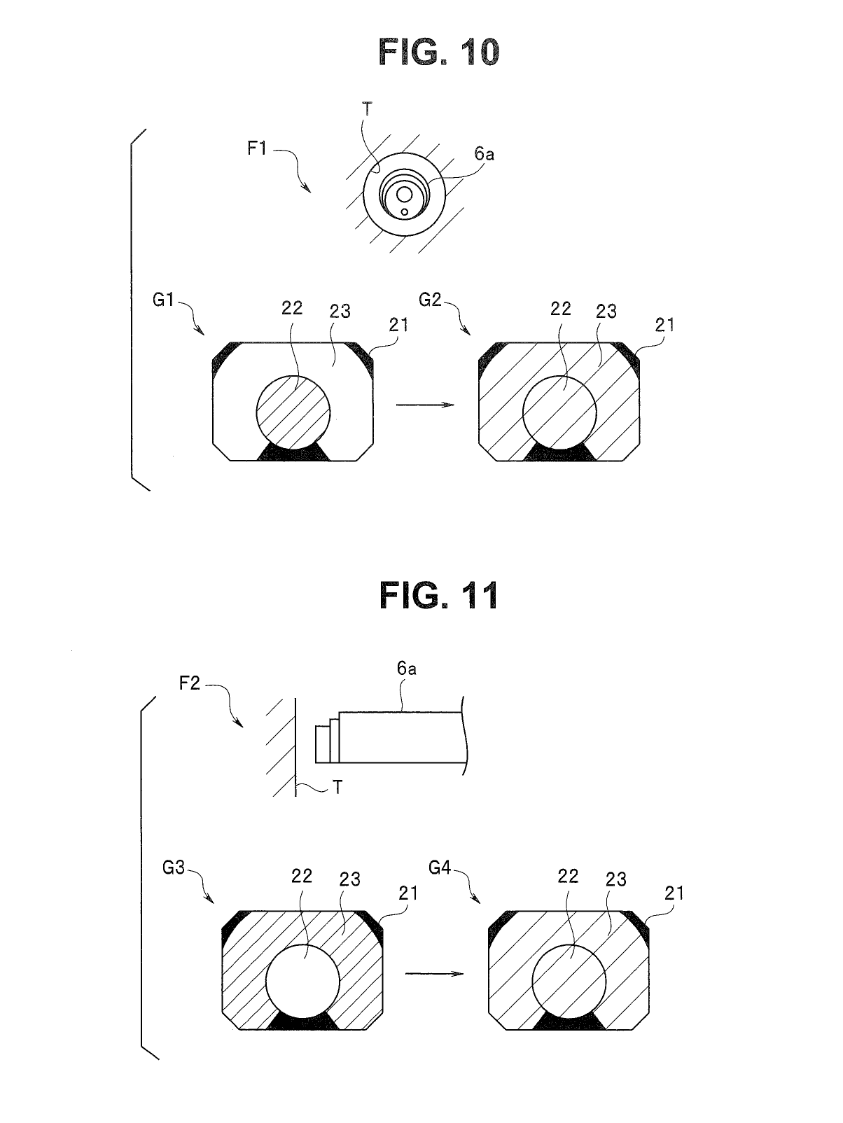

FIG. 10 is a view for describing the brightness of the endoscopic image in a case where the side face of the distal end portion 6a of the insertion portion 6 is close to an inner wall within a subject.

In the conventional apparatus, as denoted by reference character F1, when only a side face of the distal end portion 6a is too close to a surface T of in-vivo tissue of the subject, as denoted by reference character G1, only the region 23 that displays a lateral observation image in the endoscopic image 21 is bright.

In contrast, according to the present embodiment that is described above, even when only the side face of the distal end portion 6a is too close to the surface T of in-vivo tissue of the subject as denoted by reference character F1, rotation of the polarization filter 31b of the light adjustment portion 31 is controlled and the distribution of the light amount that is incident on the first region 63 and the second region 64 of the light guide 34 is changed. As a result, as denoted by reference character G2, the brightness of the region 22 that displays the front observation image and the brightness of the region 23 that displays the lateral observation image in the endoscopic image 21 can be made equal.

FIG. 11 is a view for describing the brightness of the endoscopic image in a case where a distal end face of the distal end portion 6a of the insertion portion 6 is close to an inner wall within a subject.

According to the conventional apparatus, as denoted by reference character F2, when only the distal end face of the distal end portion 6a is too close to the surface T of in-vivo tissue of the subject, as denoted by reference character G3, only the region 22 that displays a front observation image in the endoscopic image 21 is bright.

In contrast, according to the present embodiment that is described above, even when only the distal end face of the distal end portion 6a is too close to the surface T of in-vivo tissue of the subject as denoted by reference character F2, rotation of the polarization filter 31b of the light adjustment portion 31 is controlled and the distribution of the light amount that is incident on the first region 63 and the second region 64 of the light guide 34 is changed. As a result, as denoted by reference character G4, the brightness of the region 22 that displays the front observation image and the brightness of the region 23 that displays the lateral observation image in the endoscopic image 21 can be made equal.

As described above, according to the endoscope apparatus of the present embodiment, each observation image that is obtained by an endoscope that is capable of observing in two directions can be made an appropriate brightness.

### Second Embodiment

Although in the endoscope system of the first embodiment a single image pickup device picks up a subject image from both a front field of view and a lateral field of view, in the endoscope system of the present embodiment a plurality of, for example, three, image pickup devices are used, and the endoscope system is configured so that one image pickup device picks up a subject image of a front field of view, and two image pickup devices pick up subject images of two lateral fields of view, respectively, which are mutually different.

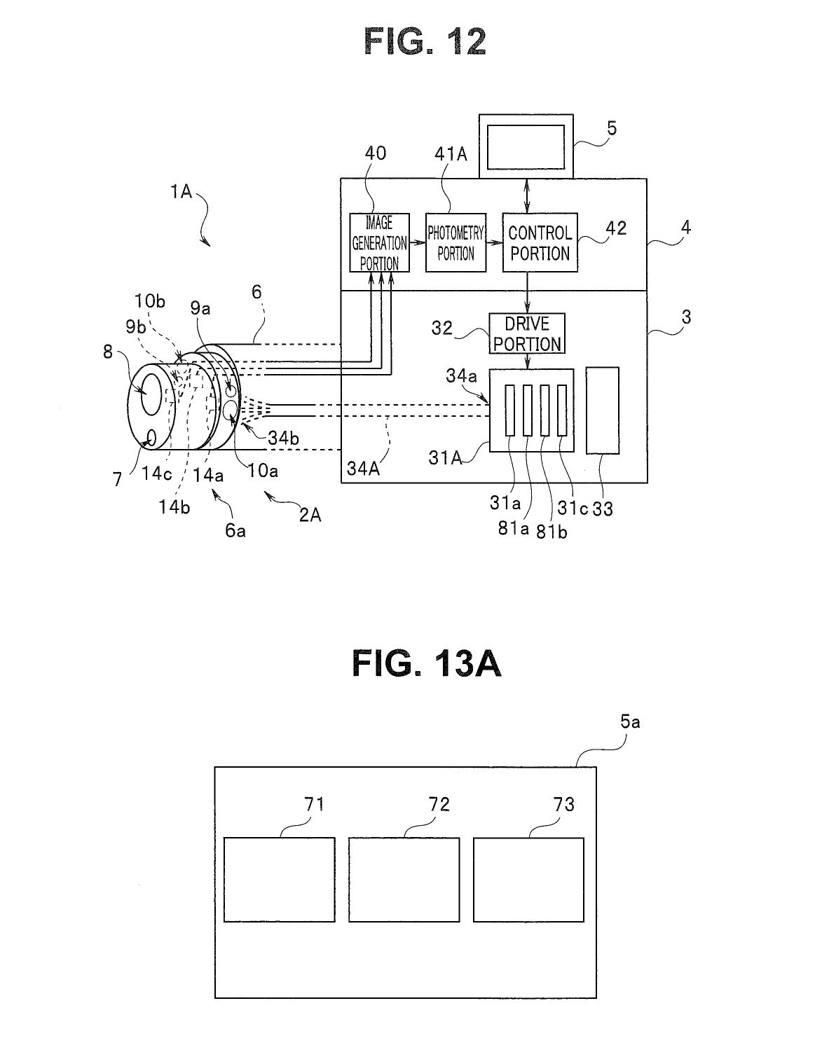

FIG. 12 is a configuration diagram that illustrates the configuration of an endoscope system relating to the present embodiment. An endoscope system 1A of the present embodiment has substantially the same configuration as the endoscope system 1 of the first embodiment, and hence components that are the same as in the endoscope system 1 are denoted by the same reference numerals and a description of such components is omitted below, and components that are different from those of the endoscope system 1 are described.

As shown in FIG. 12, in addition to the illuminating window 7, the endoscope 2A includes two illuminating windows 9a and 9b as illuminating light emitting portions, and in addition to the observation window 8, includes two observation windows 10a and 10b as subject image acquisition portions. The illuminating window 9a and observation window 10a are used for a first lateral field of view, and the illuminating window 9b and observation window 10b are used for a second lateral field of view. The two observation windows 10a and 10b are disposed at substantially equal angles in the circumferential direction of the insertion portion 6.

An image pickup unit 14a for the first lateral field of view is arranged on the rear side of the observation window 10a inside the distal end portion 6a, and an image pickup unit 14b for the second lateral field of view is arranged on the rear side of the observation window 10b inside the distal end portion 6a. An image pickup unit 14c for the front field of view is arranged on the rear side of the observation window 8 for the front field of view. The observation windows 10a and 10b are disposed at substantially equal angles in the circumferential direction of the insertion portion 6.

The three image pickup units 14a, 14b and 14c each have an image pickup device and are controlled by the processor 4, and output image pickup signals to the processor 4.

That is, the observation window 8 constitutes a subject image acquisition portion which is disposed in the distal end portion 6a of the insertion portion 6 so as to face the direction in which the insertion portion 6 is to be inserted and which is configured to acquire an image from the front as a first direction, and the observation windows 10a and 10b constitute subject image acquisition portions disposed in a side face portion of the insertion portion 6 so as to face the outer diameter direction of the insertion portion 6 and which are configured to acquire images from the lateral direction as a second direction. The image pickup unit 14c is an image pickup portion configured to photoelectrically convert an image from the observation window 8. The image pickup units 14a and 14b are image pickup portions configured to photoelectrically convert two images from the observation windows 10a and 10b.

More specifically, an image from the front as the first direction is a subject image in a front-view direction (first direction), that is, a first region of the subject, that includes the front of the insertion portion 6 that is substantially parallel to the longitudinal direction of the insertion portion 6, and an image from the lateral direction as the second direction is a subject image in a side-view direction (second direction), that is, a second region of the subject, that includes the lateral direction of the insertion portion 6 that is a direction intersecting with the longitudinal direction of the insertion portion 6. Further, the observation window 8 is a front subject image acquisition portion configured to acquire a subject image of a first region inside the subject that includes the front of the insertion portion 6, and the observation windows 10a and 10b are lateral subject image acquisition portions configured to acquire a subject image of the second region inside the subject that includes the lateral direction of the insertion portion 6.

The observation window 8 that is a subject image acquisition portion is disposed in the distal end portion 6a of the insertion portion 6 so as to face the direction in which the insertion portion 6 is to be inserted, and the observation windows 10a and 10b that are subject image acquisition portions are disposed facing the outer diameter direction of the insertion portion 6 in the side face portion of the insertion portion 6. The image pickup units 14a and 14b that are image pickup portions are disposed so as to photoelectrically convert a subject image from the observation windows 10a and 10b, respectively, with an image pickup surface, and are electrically connected to the image generation portion 40 of the processor 4 that is an image processing portion. The image pickup unit 14c that is an image pickup portion is disposed so as to photoelectrically convert a subject image from the observation window 8 with an image pickup surface, and is electrically connected to the image generation portion 40 of the processor 4 that is an image processing portion.

Hence, illuminating light for the front is emitted from the illuminating window 7, and reflected light from the subject passes through the observation window 8 and is incident on the image pickup unit 14c, and illuminating light for the sides is emitted from the two illuminating windows 9a and 9b, and reflected light from the subject passes through the observation windows 10a and 10b and is incident on the image pickup units 14a and 14b. Each of the image pickup units 14a, 14b and 14c photoelectrically converts an optical image of the subject, and outputs an image pickup signal to the processor 4.

A processing circuit such as the image generation portion 40 in the processor 4 generates three endoscopic images based on three image pickup signals from the three image pickup units 14a, 14b and 14c, and outputs the endoscopic images to the display apparatus 5.

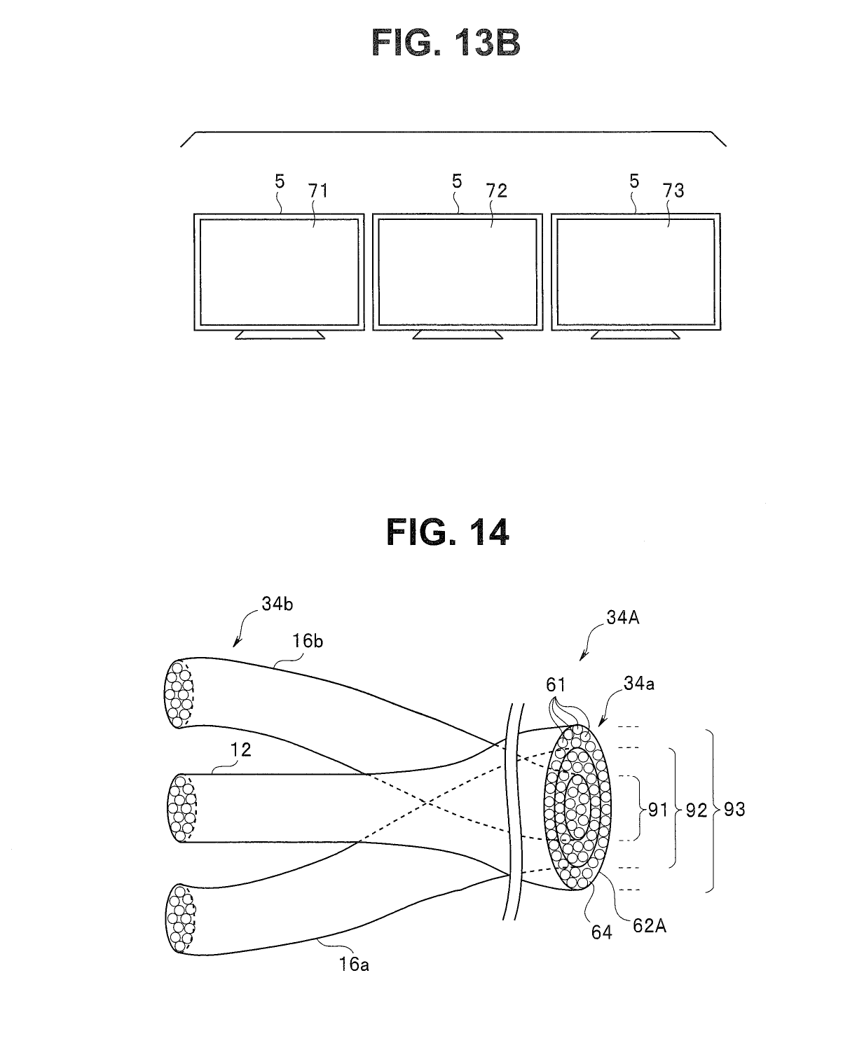

FIG. 13A is a view that illustrates an example of a display screen of endoscopic images that are displayed on the display apparatus 5.

As shown in FIG. 13A, three endoscopic images are displayed on the display screen 5a of the display apparatus 5. A first region 71 is a region that displays a first lateral observation image that is generated based on an image pickup signal from the image pickup unit 14a. The first lateral observation image corresponds to a second image that corresponds to a subject image of the second region of the subject. A second region 72 is a region that displays a front observation image that is generated based on an image pickup signal from the image pickup unit 14c. The front observation image corresponds to a second image that corresponds to a subject image of the first region of the subject. A third region 73 is a region that displays a second lateral observation image that is generated based on an image pickup signal from the image pickup unit 14b. The second lateral observation image corresponds to a second image that corresponds to a subject image of the second region of the subject.

As shown in FIG. 13A, the three endoscopic images are displayed side by side on the display screen 5a of the display apparatus 5. A photometry portion 41A of the processor 4 calculates the respective brightnesses of the three endoscopic images generated in the processor 4, and outputs the resultant values to the control portion 42.

The processor 4 is an image processing portion configured to generate an image signal that includes a front observation image and two lateral observation images. The display apparatus 5 constitutes a display portion which is configured to receive the input of an image signal from the processor 4 and to display an endoscopic image including a front observation image and two lateral observation images so that the two lateral observation images are displayed adjacent to the front observation image. In this case, a configuration is adopted in which the processor 4 displays the two lateral observation images on the display apparatus 5 so as to sandwich the front observation image therebetween. Further, although in the present embodiment the display apparatus 5 displays a plurality of images, the present invention is not limited thereto.

For example, FIG. 13B is a view illustrating an example of display screens for endoscopic images that are displayed on a plurality of the display apparatuses 5. As shown in FIG. 13B, a configuration may also be adopted in which a plurality of, as an example, three, display apparatuses 5 are adjacently disposed, in which the display apparatus 5 that displays a front observation image and the display apparatuses 5 that display lateral observation images are separately provided and are disposed so as to be adjacent to each other, and with respect to the display screens 5a of the respective display apparatuses 5, a front observation image is displayed in the second region 72 of the display apparatus 5 in the center, and lateral observation images are respectively displayed in the first region 71 and the third region 73 of the display apparatuses 5 on either side.

The light adjustment portion 31A includes three polarization filters 31a, 81a and 81b as a light amount adjustment portion, and the diaphragm 31c. The configuration of the three polarization filters 31a, 81a and 81b is described later. The polarization filters 81a and 81b are rotationally controlled by the control portion 42.

The amount of light from the light source 33 is adjusted by the diaphragm 31c at the light adjustment portion 31A, and the balance of the light amount is adjusted by the three polarization filters 31a, 81a and 81b so that the brightnesses of the respective images displayed in the regions 71, 72 and 73 are appropriate. Light emitted from the light adjustment portion 31A is condensed in the proximal end portion 34a of the light guide 34A by an unshown light condensing apparatus. The light that is emitted from the light adjustment portion 31A passes through the light guide 34A, and is emitted from the distal end portion 34b of the light guide 34A.

The light guide 34A includes the light guide for front illumination 12, a first light guide for lateral illumination 16a and a second light guide for lateral illumination 16b.

FIG. 14 is a schematic view that illustrates the configuration of the light guide 34A. The light guide 34A is constituted by bundling a large number of optical fibers 61. The proximal end portion 34a of the light guide 34A has a circular incident surface 62A on which light that passed through the light adjustment portion 31A is incident. End faces of the large number of optical fibers 61 are gathered together at the incident surface 62A that constitutes a light-receiving portion.

The incident surface 62A has three regions 91, 92 and 93 that are light-receiving regions. An optical fiber group having end portions in the first region 91 is the second light guide for lateral illumination 16b. An optical fiber group having end portions in the second region 92 is the first light guide for lateral illumination 16a. An optical fiber group having end portions in the third region 93 is the light guide for front illumination 12.

In FIG. 14, the proximal end portions of the second light guide for lateral illumination 16b are held together and disposed in the circular first region 91 at the center part of the circular incident surface 62A of the proximal end portion 34a of the light guide 34. The proximal end portions of the first light guide for lateral illumination 16a are held together and disposed in the circular ring-shaped region 92 around the circular region 91 at the center part of the circular incident surface 62A of the proximal end portion 34a of the light guide 34. The proximal end portions of the light guide for front illumination 12 are held together and disposed in the circular ring-shaped region 93 around the region 92 of the proximal end portion 34a of the light guide 34.

Note that a partition film may be provided between the light guide for front illumination 12 and the light guide for lateral illumination 16a so that light does not leak, and may also be provided between the light guide for lateral illumination 16a and the light guide for lateral illumination 16b so that light does not leak therebetween.

The respective configurations of the two polarization filters 81a and 81b of the light adjustment portion 31A will now be described. As shown in FIG. 5, the polarization filter 31a is a circular polarization filter in which slits in the lengthwise direction are provided in the entire area R0, and which is fixed so as not to rotate.

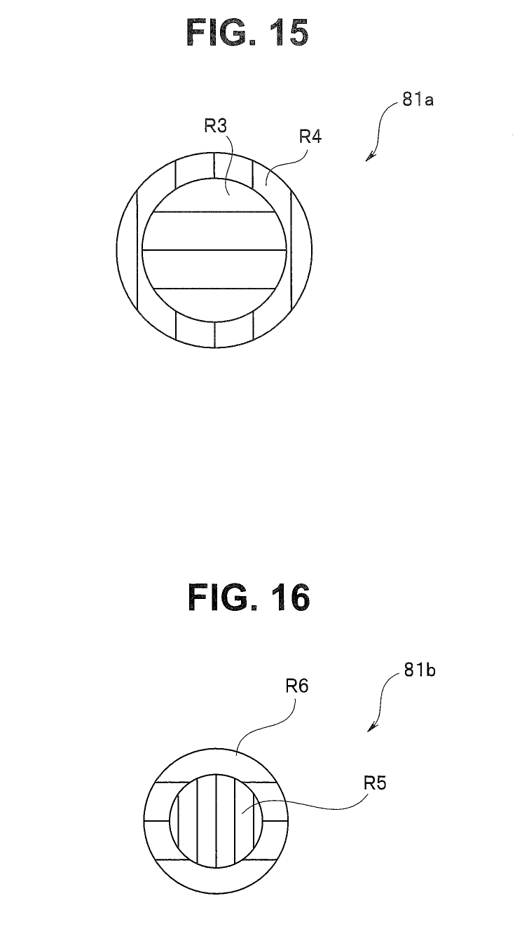

FIG. 15 is a view that illustrates the configuration of the polarization filter 81a. The polarization filter 81a is a circular polarization filter in which crosswise slits having the same width as the slits of the polarization filter 31a are provided in a central circular area R3, and lengthwise slits having the same width as the slits of the polarization filter 31a are provided in a circular ring-shaped area R4 around the circular area R3, and which is disposed so as to be rotatable around the central axis of the circle. That is, the direction of the slits in the area R3 and the direction of the slits in the area R4 are orthogonal. In the case illustrated in FIG. 15, light which passes through the area R3 is light which vibrates in the crosswise direction, and light which passes through the area R4 is light which vibrates in the lengthwise direction.

Rotating of the polarization filter 81a is performed by the drive portion 32 under control of the control portion 42.

FIG. 16 is a view that illustrates the configuration of the polarization filter 81b. The polarization filter 81b is a circular polarization filter in which lengthwise slits having the same width as the slits of the polarization filter 31a are provided in a central circular area R5, and crosswise slits having the same width as the slits of the polarization filter 31a are provided in a circular ring-shaped area R6 around the circular area R5, and which is disposed so as to be rotatable around the central axis of the circle. That is, the direction of the slits in the area R5 and the direction of the slits in the area R6 are orthogonal. In the case illustrated in FIG. 16, light which passes through the area R5 is light which vibrates in the lengthwise direction, and light which passes through the area R6 is light which vibrates in the crosswise direction.

Rotating of the polarization filter 81b is performed by the drive portion 32 under control of the control portion 42. Hence, the control portion 42 performs rotational control of the polarization filters 81a and 81b individually.

The polarization filters 31a, 81a and 81b are disposed on the same axis as the diaphragm 31c. The amount of light from the light source 33 is adjusted by the diaphragm 31c. Light that passed through the diaphragm 31c is transmitted through the polarization filter 81b and is incident on the polarization filter 81a, and light that is transmitted through the polarization filter 81a is incident on the polarization filter 31a and is then incident on the incident surface 62A of the proximal end portion 34a of the light guide 34.

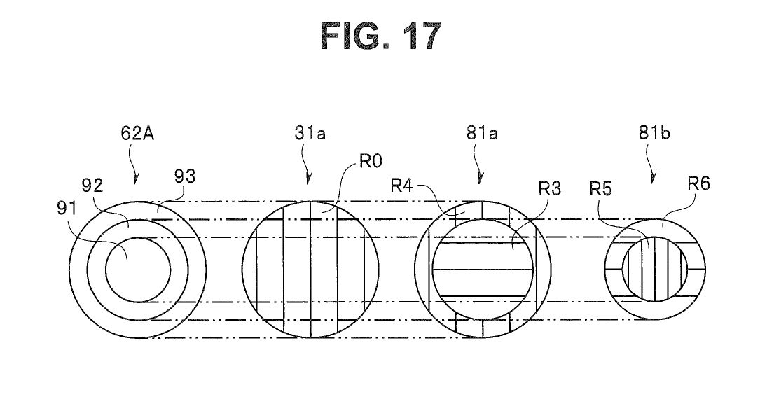

FIG. 17 is a view that illustrates the correspondence relation between the three regions 91, 92 and 93 of the proximal end portion 34a of the light guide 34A, and the respective areas R3, R4, R5 and R6 of the three polarization filters 31a, 81a and 81b.

As shown in FIG. 17, the outer diameters of the polarization filters 31a and 81a are equal to the outer diameter of the region 93 of the incident surface 62A of the light guide 34. The outer diameter of the area R3 of the polarization filter 81a is equal to the outer diameter of the region 92 of the incident surface 62A.

In addition, the outer diameter of the area R3 of the polarization filter 81a, the outer diameter of the area R6 of the polarization filter 81b, and the outer diameter of the region 92 of the incident surface 62A of the light guide 34 are equal. The outer diameter of the area R5 of the polarization filter 81b and the outer diameter of the region 91 of the incident surface 62A are equal.

The respective polarization filters 31a, 81a and 81b are disposed relative to the incident surface 62A of the light guide 34A so that light emitted from the area R4 of the polarization filter 81a passes through the polarization filter 31a and is incident on the third region 93 of the incident surface 62A.

The respective polarization filters 31a, 81a and 81b are disposed relative to the incident surface 62A of the light guide 34A so that light emitted from the area R6 of the polarization filter 81b passes through the area R3 of the polarization filter 81a and further passes through the polarization filter 31a and is incident on the second region 92 of the incident surface 62A.

The respective polarization filters 31a, 81a and 81b are disposed relative to the incident surface 62A of the light guide 34A so that light emitted from the area R5 of the polarization filter 81b passes through the area R3 of the polarization filter 81a and further passes through the polarization filter 31a and is incident on the first region 91 of the incident surface 62A.

By rotating the polarization filter 81a within a range from 0 degrees to 90 degrees relative to the polarization filter 31a, the illumination for a front observation image and the entire light amount of illumination for lateral observation images can be balanced.

In addition, the illumination for a front observation image and the entire light amount of illumination for a lateral observation image are balanced, the illumination for a first lateral observation image and the illumination for a second lateral observation image can be balanced by rotating the polarization filter 81b within a range from 0 degrees to 90 degrees relative to the polarization filter 81a.

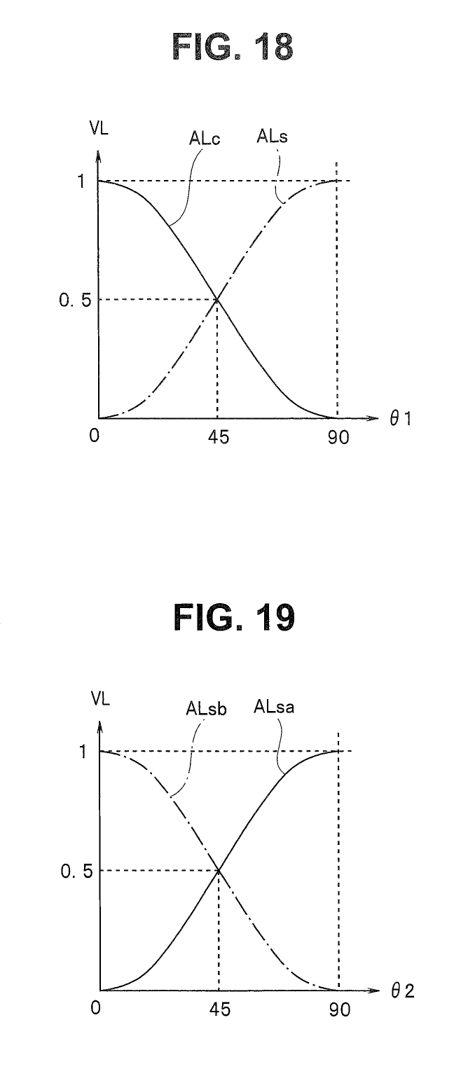

FIG. 18 is a graph illustrating the relation between a rotational angle θ1 of the polarization filter 81a with respect to the polarization filter 31a, and a light amount VL that is incident on the region 93 and a light amount VL that is incident on the region 92 of the light guide 34.

In this case, the rotational angle θ1 of the polarization filter 81a with respect to the polarization filter 31a when the direction of the slits in the polarization filter 31a and the direction of the slits in the area R4 of the polarization filter 81a are parallel is taken as 0 degrees.

When the rotational angle θ1 of the polarization filter 81a with respect to the polarization filter 31a changes from 0 degrees toward 90 degrees, the light amount VL incident on the region 93 of the light guide 34 gradually decreases as shown by a solid line ALc, and the light amount VL incident on the two regions 91 and 92 of the light guide 34 gradually increases as shown by an alternate long and short dashed line ALs.

When the rotational angle θ1 of the polarization filter 81a is 0 degrees, as shown in FIG. 18, the light amount VL incident on the region 93 is 1 (that is, 100% transmission) and the light amount VL incident on the regions 91 and 92 is 0 (that is, 0% transmission). When the rotational angle θ1 of the polarization filter 81a is 90 degrees, as shown in FIG. 18, the light amount VL incident on the region 93 is 0 (that is, 0% transmission) and the light amount VL incident on the regions 91 and 92 is 1 (that is, 100% transmission).

When the rotational angle θ1 of the polarization filter 81a is 45 degrees, the light amount VL incident on the region 93 and the light amount VL incident on the regions 91 and 92 are each 0.5 (that is, 50% transmission).

Thus, by changing the rotational angle θ1 of the polarization filter 81a within the range from 0 to 90 degrees, the distribution between the light amount incident on the region 93 and the light amount incident on the two regions 91 and 92 of the light guide 34 can be changed.

FIG. 19 is a graph illustrating the relation between a rotational angle θ2 of the polarization filter 81b with respect to the polarization filter 81a, and a light amount incident on the region 92 and a light amount incident on the region 91 of the light guide 34.

In this case, the rotational angle θ2 of the polarization filter 81b with respect to the polarization filter 81a when the direction of the slits in the area R3 of the polarization filter 81a and the direction of the slits in the area R6 of the polarization filter 81b are parallel is taken as 0 degrees.

When the rotational angle θ2 of the polarization filter 81b with respect to the polarization filter 81a changes from 0 degrees toward 90 degrees, the light amount VL incident on the region 92 of the light guide 34 gradually decreases as shown by an alternate long and short dashed line ALsb, and the light amount VL incident on the region 91 the light guide 34 gradually increases as shown by, a solid line ALsa.

When the rotational angle θ2 of the polarization filter 81b with respect to the polarization filter 81a is 0 degrees, as shown in FIG. 19, the light amount VL incident on the region 92 is 1 (that is, 100% transmission) and the light amount VL incident on the region 91 is 0 (that is, 0% transmission). When the rotational angle θ2 of the polarization filter 81b is 90 degrees, as shown in FIG. 19, the light amount VL incident on the region 92 is 0 (that is, 0% transmission) and the light amount VL incident on the region 91 is 1 (that is, 100% transmission).

When the rotational angle θ2 of the polarization filter 81b is 45 degrees, the light amount incident on the region 92 and the light amount incident on the region 91 are each 0.5 (that is, 50% transmission).

Thus, by changing the rotational angle θ2 of the polarization filter 81b within the range from 0 to 90 degrees, the distribution between the light amount incident on the region 92 and the light amount incident on the region 91 of the light guide 34 can be changed.

Next, operations of the processor 4 are described.

As described above, the photometry portion 41A calculates brightnesses La1, La21, La22 of the respective images of the regions 71, 72 and 73 in an endoscopic image, and outputs the calculated brightness values to the control portion 42. The brightnesses of the respective regions are average values of the luminance of all pixels within the respective regions. The control portion 42 as a brightness comparison portion drives the drive portion 32 to rotate the polarization filter 81a so that the brightness La1 of the image of the region 72 that displays a front observation image and a brightness La2 (average value of the brightnesses La21 and La22) of the two images of the region 71 that displays a first lateral observation image and the region 73 that displays a second lateral observation image becomes equal.

Rotational control of the polarization filter 81a is performed by feedback control that, for example, while monitoring the brightnesses La1 and La2, drives the drive portion 32 within a range in which the rotational angle θ1 is from 45 degrees to 90 degrees when the brightness La1 is greater than the brightness La2, and drives the drive portion 32 within a range in which the rotational angle θ1 is from 0 degrees to 45 degrees when the brightness La1 is less than the brightness La2, so that the brightnesses La1 and La2 thus become equal.

In addition, after the brightnesses La1 and La2 become equal, the control portion 42 drives the drive portion 32 to rotate the polarization filter 81b so that the brightness La21 of the image of the region 71 and the brightness La22 of the image of the region 73 become equal.

Rotational control of the polarization filter 81b is performed by feedback control that, for example, while monitoring the brightnesses La21 and La22, drives the drive portion 32 within a range in which the rotational angle θ2 is from 0 degrees to 45 degrees when the brightness La21 is greater than the brightness La22, and drives the drive portion 32 within a range in which the rotational angle θ2 is from 45 degrees to 90 degrees when the brightness La21 is less than the brightness La22, so that the brightnesses La21 and La22 thus become equal.

As described in the foregoing, according to the endoscope apparatus of the present embodiment, each observation image obtained by an endoscope that is capable of observing in three directions can be made an appropriate brightness.

### (Modification)

A modification of the configuration of the three polarization filters in the endoscope apparatus that is capable of observing in three directions of the second embodiment will now be described.

Although in the above described second embodiment, slits are provided over the entire region of each polarization filter, in the present modification a region in which slits are not formed at one part is provided in one polarization filter.

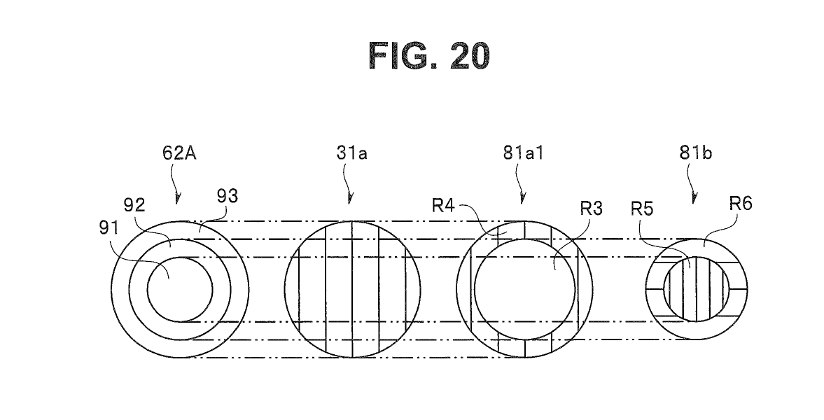

FIG. 20 is a view illustrating a correspondence relation between the three regions 91, 92 and 93 of the proximal end portion of the light guide 34A and three polarization filters 31a, 81a1 and 81b as a light amount adjustment portion. Although slits having the same width as slits of the polarization filter 31a are provided in an area 4 of the polarization filter 81a1 that is disposed between the polarization filters 31a and 81b, slits are not provided in an area R3 of the polarization filter 81a1. The remaining configuration of the three polarization filters 31a, 81a1 and 81b is the same as that of the polarization filters 31a, 81a and 81b of the second embodiment that is described above. The plurality of polarization filters as an example of a light amount adjustment portion have, for example, respective portions that are disposed substantially collinearly so as to lie along the optical axis of the illuminating light.

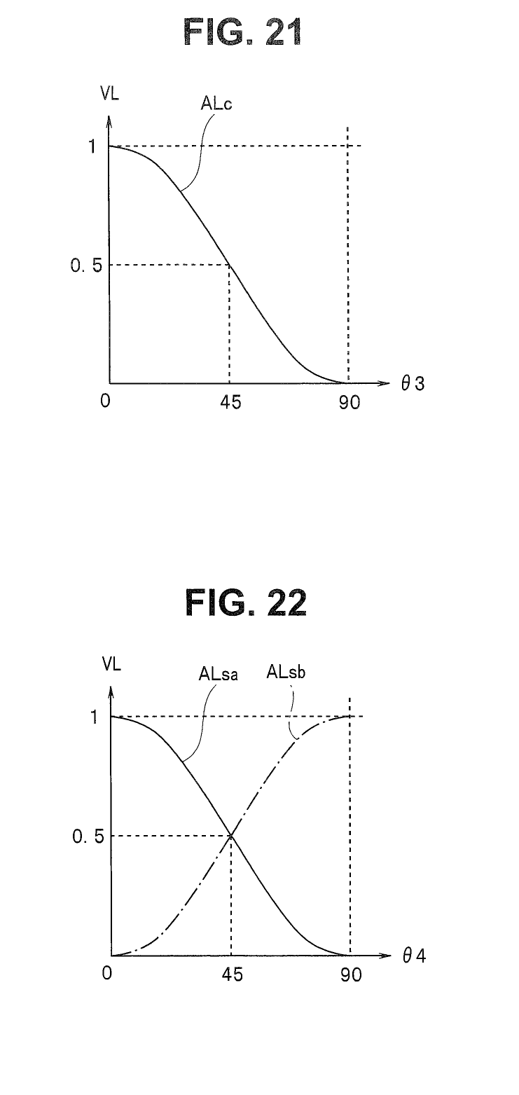

FIG. 21 is a graph illustrating the relation between a rotational angle θ3 of the polarization filter 81a1 with respect to the polarization filter 31a, and a light amount VL that is incident on the region 93 of the light guide 34A.

In this case, the rotational angle θ3 of the polarization filter 81a1 with respect to the polarization filter 31a when the direction of the slits in the area R4 of the polarization filter 31a and the direction of the slits in the area R4 of the polarization filter 81a1 are parallel is taken as 0 degrees.

When the rotational angle θ3 of the polarization filter 81a1 with respect to the polarization filter 31a changes from 0 degrees toward 90 degrees, the light amount VL incident on the region 93 of the light guide 34A gradually decreases as shown by a solid line ALc.

When the rotational angle θ3 of the polarization filter 81a1 is 0 degrees, as shown in FIG. 21, the light amount VL incident on the region 93 is 1 (that is, 100% transmission). When the rotational angle θ3 of the polarization filter 81a1 is 90 degrees, as shown in FIG. 21, the light amount VL incident on the region 93 is 0 (that is, 0% transmission).

When the rotational angle θ3 of the polarization filter 81a1 is 45 degrees, light amount VL incident on the region 93 is 0.5 (that is, 50% transmission).

Thus, by changing the rotational angle θ3 of the polarization filter 81a1 within the range from 0 to 90 degrees, the light amount incident on the region 93 of the light guide 34A can be changed.

FIG. 22 is a graph illustrating the relation between a rotational angle θ4 of the polarization filter 81b with respect to the polarization filter 31a, and a light amount that is incident on the region 92 and a light amount that is incident on the region 91 of the light guide 34A.

In this case, the rotational angle θ4 of the polarization filter 81b with respect to the polarization filter 31a when the direction of the slits in the area R0 of the polarization filter 31a and the direction of the slits in the area R6 of the polarization filter 81b are orthogonal is taken as 0 degrees.

When the rotational angle θ4 of the polarization filter 81b with respect to the polarization filter 31a changes from 0 degrees toward 90 degrees, the light amount VL incident on the region 92 of the light guide 34A gradually increases as shown by a solid line ALsb and the light amount VL incident on the region 91 of the light guide 34A gradually decreases as shown by an alternate long and short dashed line ALsa.

When the rotational angle θ4 of the polarization filter 81b with respect to the polarization filter 31a is 0 degrees, as shown in FIG. 22, the light amount VL that is incident on the region 92 is 0 (that is, 0% transmission) and the light amount VL that is incident on the region 91 is 1 (that is, 100% transmission). When the rotational angle θ4 of the polarization filter 81b is 90 degrees, as shown in FIG. 22, the light amount VL that is incident on the region 92 is 1 (that is, 100% transmission) and the light amount VL that is incident on the region 91 is 0 (that is, 0% transmission).

When the rotational angle θ4 of the polarization filter 81b is 45 degrees, the light amount VL that is incident on the region 92 and the light amount VL that is incident on the region 91 are each 0.5 (that is, 50% transmission).

Thus, by changing the rotational angle θ4 of the polarization filter 81b with respect to the polarization filter 31a within the range from 0 to 90 degrees, the distribution between the light amount incident on the region 92 and the light amount incident on the region 91 of the light guide 34A can be changed.

In the present modification, the control portion 42a light amount for front illumination is adjusted by controlling the rotational angle θ3 of the polarization filter 81a1 in accordance with the brightness of an image of the region 72, and the balance between the brightness of an image of the region 71 and the brightness of an image of the region 73 can be adjusted by controlling the rotational angle θ4 of the polarization filter 81b.

As described in the foregoing, by means of the endoscope apparatus of the modification of the present embodiment also, each observation image obtained by an endoscope that is capable of observing in three directions can be made an appropriate brightness.

### Third Embodiment

Although an endoscope system of the present embodiment is, similarly to the second embodiment, also a system in which three image pickup devices are used and which is configured to receive a subject image of one front field of view and subject images of two lateral fields of view, the endoscope system of the present embodiment has a different configuration to the second embodiment.

In the endoscope system of the present embodiment, the configuration of a light adjustment portion is different to the second embodiment.

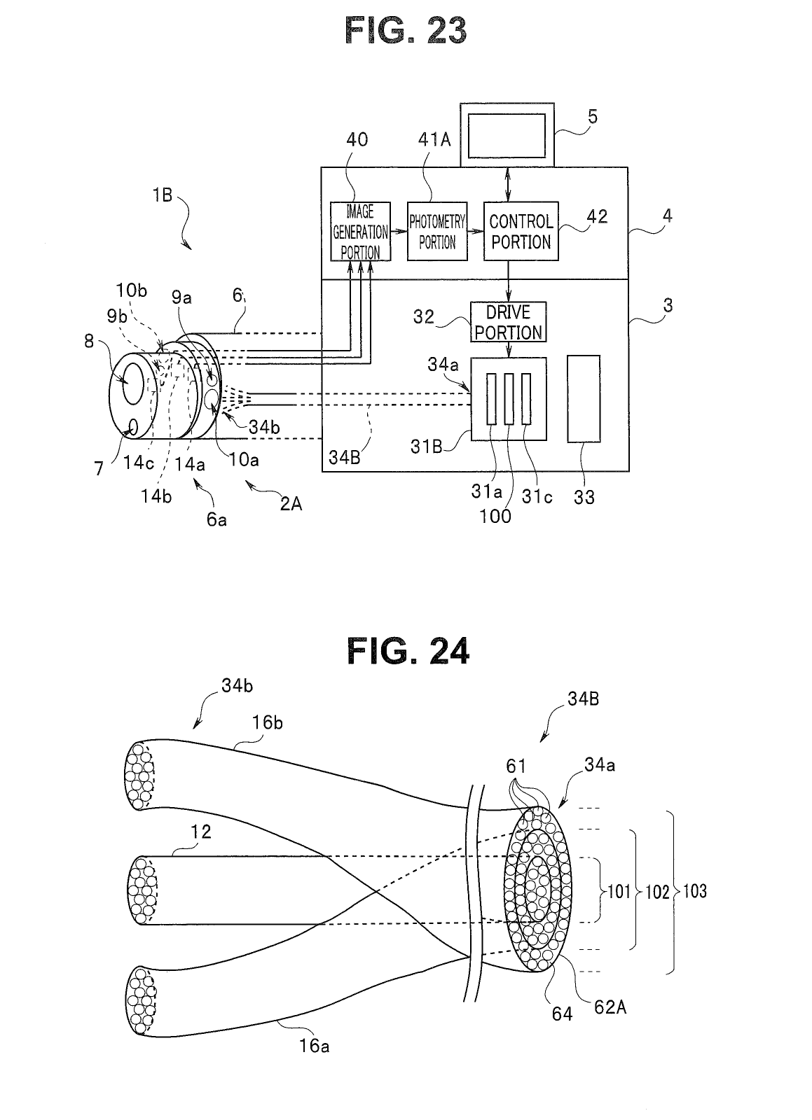

FIG. 23 is a configuration diagram that illustrates the configuration of the endoscope system relating to the present embodiment. An endoscope system 1B of the present embodiment has substantially the same configuration as the endoscope system 1 of the first embodiment, and hence components that are the same as in the endoscope system 1 are denoted by the same reference numerals and a description of such components is omitted below, and components that are different from those of the endoscope system 1 will be described.

As shown in FIG. 23, a light adjustment portion 31B includes the polarization filter 31a and a polarization filter 100 as a light amount adjustment portion and the diaphragm 31c.

FIG. 24 is a schematic view that illustrates the configuration of a light guide 34B relating to the present embodiment. The light guide 34B is constituted by bundling a large number of optical fibers 61. A proximal end portion 34a of the light guide 34B has a circular incident surface 62A on which light that passed through the light adjustment portion 31B is incident. End faces of the large number of optical fibers 61 are gathered together at the incident surface 62A.

The incident surface 62A has three regions 101, 102 and 103 that are light-receiving regions. An optical fiber group having end portions in the first region 101 is the light guide for front illumination 12. An optical fiber group having end portions in the second region 102 is the first light guide for lateral illumination 16a. An optical fiber group having end portions in the third region 103 is the second light guide for lateral illumination 16b.

The plurality of polarization filters as an example of a light amount adjustment portion have, for example, respective portions that are disposed substantially collinearly so as to lie along the optical axis of the illuminating light.

In FIG. 24, the proximal end portions of the light guide for front illumination 12 are held together and disposed in the circular first region 101 at the center part of the circular incident surface 62A of the proximal end portion 34a of the light guide 34B. The proximal end portions of the first light guide for lateral illumination 16a are held together and disposed in the circular ring-shaped region 102 around the circular region 101 at the center part of the circular incident surface 62A of the proximal end portion 34a of the light guide 34B. The proximal end portions of the second light guide for lateral illumination 16b are held together and disposed in the circular ring-shaped region 103 around the region 102 of the proximal end portion 34a of the light guide 34B.

Note that a partition film may be provided between the light guide for front illumination 12 and the light guide for lateral illumination 16a so that light does not leak, and may also be provided between the light guide for lateral illumination 16a and the light guide for lateral illumination 16b so that light does not leak.

The respective configurations of the two polarization filters 31a and 100 of the light adjustment portion 31B will now be described. As shown in FIG. 5, the polarization filter 31a is a circular polarization filter in which slits in the lengthwise direction are provided in the entire area R0, and which is fixed so as not to rotate.

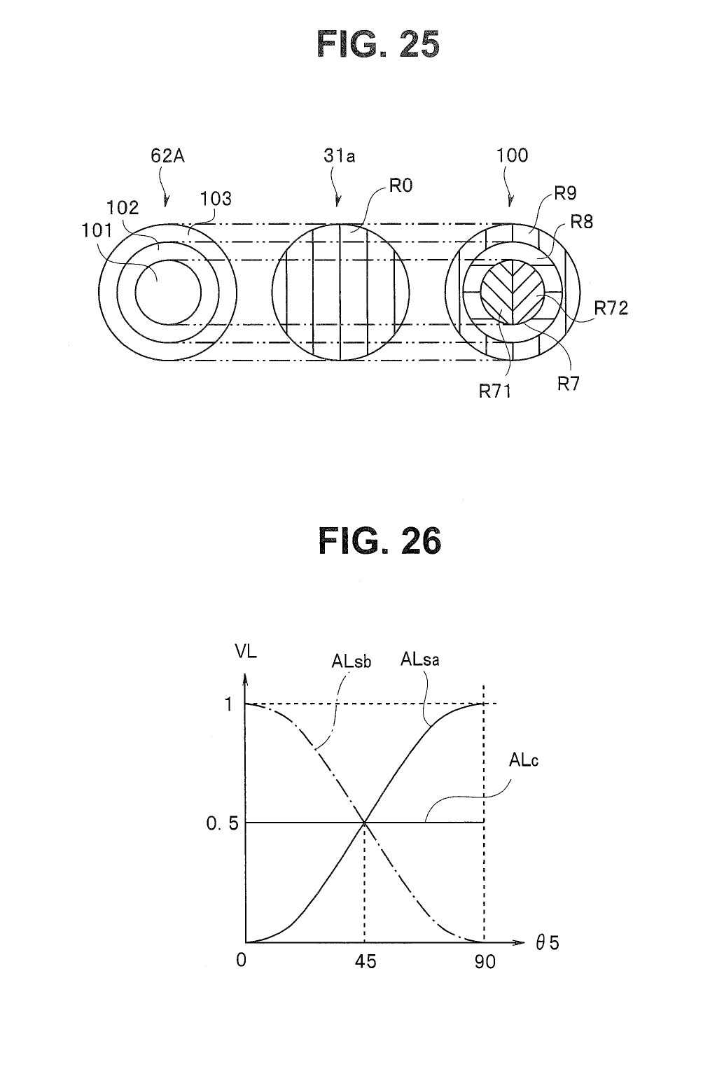

FIG. 25 is a view that illustrates the configuration of the polarization filter 100, and also illustrates the correspondence relation between the three regions 101, 102 and 103 of the proximal end portion of the light guide 34B and respective areas R0, R′ (R71, R72), R8 and R9 of the polarization filter 31a and the polarization filter 100.

The polarization filter 100 has a circular area R7 at a center part, and has two semicircular areas R71 and R72 inside the area R7. Slits in a diagonal direction that have the same width as the slits of the polarization filter 31a are provided in the areas R71 and R72. The area R7 has the two regions 71 and 72 in which the direction of the slits (that is, the polarization direction) in the area R71 and the direction of the slits (the polarization direction) of the area R72 are orthogonal to each other. The direction of the slits in the area R71 and the direction of the slits in the area R72 are each at an angle of 45 degrees with respect to the direction of the slits of the polarization filter 31a.

Crosswise slits having the same width as the slits of the polarization filter 31a are provided in the circular ring-shaped area R8 around the central circular area R7. Lengthwise slits having the same width as the slits of the polarization filter 31a are provided in the circular ring-shaped area R9 around the circular ring-shaped area R8. The circular polarization filter 100 is disposed so as to be rotatable around the central axis of the circle. In the case of the state illustrated in FIG. 25, light that passes through the area R71 is light which vibrates in a diagonal 45-degree direction relative to the direction of the slits of the polarization filter 31a, and light that passes through the area R72 is light which, relative to the direction of the slits of the polarization filter 31a, vibrates in a diagonal 45-degree direction that is opposite to the diagonal 45-degree direction of the light that passes through the area R71.

Rotating of the polarization filter 100 is performed by the drive portion 32 under control of the control portion 42.

The polarization filter 31a and the polarization filter 100 are disposed on the same axis as the diaphragm 31c. The amount of light from the light source 33 is adjusted by the diaphragm 31c. Light that passed through the diaphragm 31c is transmitted through the polarization filter 100 and is incident on the polarization filter 31a, and is thereafter incident on the incident surface 62A of the proximal end portion 34a of the light guide 34B.

As shown in FIG. 25, the outer diameters of the polarization filter 31a and the polarization filter 100 are equal to the outer diameter of the region 103 of the incident surface 62A of the light guide 34B. The outer diameter of the area R8 of the polarization filter 100 and the outer diameter of the region 102 of the incident surface 62A are equal.

In addition, the outer diameter of the area R7 of the polarization filter 100 and the outer diameter of the region 101 of the incident surface 62A of the light guide 34B are equal.

The polarization filter 31a and the polarization filter 100 are disposed with respect to the incident surface 62A of the light guide 34B so that light emitted from the area R9 of the polarization filter 100 is transmitted through the polarization filter 31a and is incident on the third region 103 of the incident surface 62A.

The polarization filter 31a and the polarization filter 100 are disposed with respect to the incident surface 62A of the light guide 34B so that light emitted from the area R8 of the polarization filter 100 is transmitted through the polarization filter 31a and is incident on the second region 102 of the incident surface 62A.

The polarization filter 31a and the polarization filter 100 are disposed with respect to the incident surface 62A of the light guide 34B so that light emitted from the area R7 of the polarization filter 100 is transmitted through the polarization filter 31a and is incident on the region 101 of the incident surface 62A.

By rotating the polarization filter 100 within a range of 0 degrees to 90 degrees relative to the polarization filter 31a, the two amounts of illumination light for a lateral observation image can be balanced while keeping the light amount of the illumination for a front observation image constant.

FIG. 26 is a graph illustrating the relation between a rotational angle θ5 of the polarization filter 100 with respect to the polarization filter 31a, and light amounts that are incident on the respective regions of the light guide 34B.

In this case, the rotational angle θ5 of the polarization filter 100 with respect to the polarization filter 31a when the direction of the slits in the area R0 of the polarization filter 31a and the direction of the slits in the area R8 of the polarization filter 100 are orthogonal is taken as 0 degrees.

When the rotational angle θ5 of the polarization filter 100 with respect to the polarization filter 31a changes from 0 degrees toward 90 degrees, the light amount VL incident on the region 101 of the light guide 34B is constant as shown by a solid line ALc. This is because the directions of the slits of the areas R71 and R72 are orthogonal to each other.

When the rotational angle θ5 of the polarization filter 100 with respect to the polarization filter 31a changes from 0 degrees toward 90 degrees, the light amount VL incident on the region 102 of the light guide 34B gradually increases as shown by a solid line ALsa, and the light amount VL incident on the region 103 of the light guide 34B gradually decreases as shown by an alternate long and short dashed line ALsb.

When the rotational angle θ5 of the polarization filter 100 with respect to the polarization filter 31a is 0 degrees, as shown in FIG. 26, the light amount VL that is incident on the region 102 is 0 (that is, 0% transmission) and the light amount VL incident on the region 103 is 1 (that is, 100% transmission). When the rotational angle θ5 of the polarization filter 100 is 90 degrees, as shown in FIG. 26, the light amount VL that is incident on the region 102 is 1 (that is, 100% transmission) and the light amount VL incident on the region 103 is 0 (that is, 0% transmission).

When the rotational angle θ5 of the polarization filter 100 is 45 degrees, the light amount VL that is incident on the region 102 and the light amount VL that is incident on the region 103 are each 0.5 (that is, 50% transmission).

Thus, by changing the rotational angle θ5 of the polarization filter 100 with respect to the polarization filter 31a within the range from 0 to 90 degrees, the distribution between the light amount incident on the region 102 and the light amount incident on the region 103 of the light guide 34B can be changed.

As described in the foregoing, by means of the endoscope apparatus of the present embodiment also, each observation image obtained by an endoscope that is capable of observing in three directions can be made an appropriate brightness.

### (Modification)

A modification of the configuration of two polarization filters in the endoscope apparatus capable of observing three directions of the third embodiment will now be described.

Although in the above described third embodiment the incident surface 62A of the light guide 34B has a semicircular region and a circular ring-shaped region, in the present modification the circular incident surface 62A of the light guide 34B has a layer-like region 104 at the center and has two regions 105 and 106 which are formed in a manner that sandwiches the region 104 therebetween.

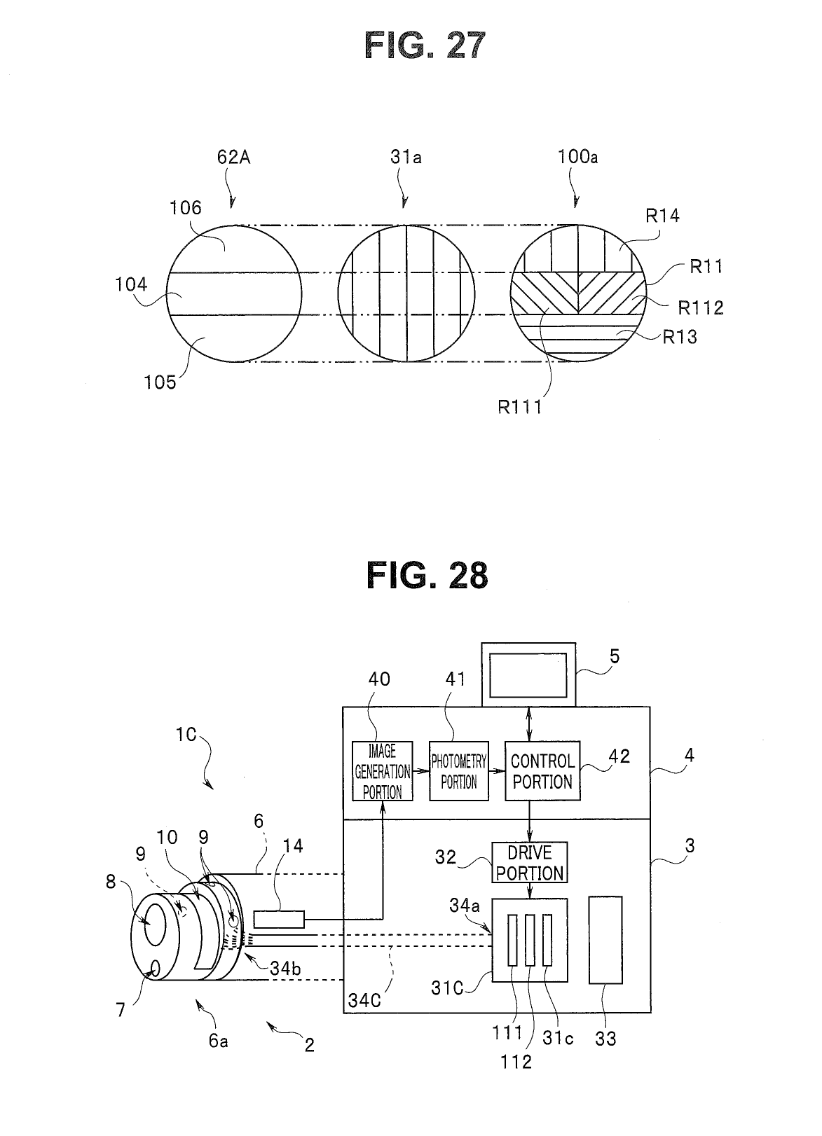

FIG. 27 is a view illustrating the correspondence relation between three regions 104, 105 and 106 of the incident surface 62A of the light guide 34B and two polarization filters 31a and 100a as a light amount adjustment portion. An area R11 of the polarization filter 100a has the same shape as the region 104 of the incident surface 62A, and has two areas R111 and R112 which have the center part of the area R11 as a boundary therebetween.

Slits in a diagonal direction that have the same width as slits of the polarization filter 31a are provided in the areas R111 and R112. The direction of the slits in the area R111 and the direction of the slits in the area R112 are orthogonal to each other. In the case of the state shown in FIG. 27, the direction of the slits in the area R111 and the direction of the slits in area R112 are each at an angle of 45 degrees relative to the direction of the slits of the polarization filter 31a.

The circular polarization filter 31a is disposed so as to be rotatable around the central axis of the circle, and the polarization filter 100a is fixed with respect to the light guide 34B and does not rotate. In the case of the state illustrated in FIG. 27, light that passes through the area R111 is light which vibrates in a diagonal 45-degree direction relative to the direction of the slits of the polarization filter 31a, and light that passes through the area R112 is light which, relative to the direction of the slits of the polarization filter 31a, vibrates in a diagonal 45-degree direction that is opposite to the diagonal 45-degree direction of the light that passes through the area R111.

In the layer-like area R13 on the lower side in FIG. 27, crosswise slits are provided that have the same width as the slits in the polarization filter 31a. In the layer-like area R14 on the upper side in FIG. 27, lengthwise slits are provided that have the same width as the slits in the polarization filter 31a. That is, the direction of the slits in the area R13 and the direction of the slits in the area R14 are orthogonal to each other.

The respective polarization filters 31a and 100a are disposed with respect to the incident surface 62A of the light guide 34B so that light emitted from the area R11 of the polarization filter 100a passes through the polarization filter 31a and is incident on the region 104 of the incident surface 62A.

The respective polarization filters 31a and 100a are disposed with respect to the incident surface 62A of the light guide 34B so that light emitted from the area R13 of the polarization filter 100a passes through the polarization filter 31a and is incident on the region 105 of the incident surface 62A.

Likewise, the respective polarization filters 31a and 100a are disposed with respect to the incident surface 62A of the light guide 34B so that light emitted from the area R14 of the polarization filter 100a passes through the polarization filter 31a and is incident on the region 106 of the incident surface 62A.

An optical fiber group having end portions in the first region 104 is an end face of the light guide for front illumination 12. An optical fiber group having end portions in the second region 105 is the first light guide for lateral illumination 16a, and an optical fiber group having end portions in the third region 105 is the second light guide for lateral illumination 16b.

Rotating of the polarization filter 31a is performed by the drive portion 32 under control of the control portion 42.

Even if the polarization filter 31a rotates, a light amount VL that is incident on the region 104 of the light guide 34B is constant. This is because the directions of the slits of the areas R111 and R112 are orthogonal to each other.

When a rotational angle θ6 of the polarization filter 31a with respect to the polarization filter 100s changes from 0 degrees toward 90 degrees, as shown in FIG. 26, the light amount VL incident on the region 105 of the light guide 34B gradually increases as shown by a solid line ALsa, and the light amount VL incident on the region 106 of the light guide 34B gradually decreases as shown by an alternate long and short dashed line ALsb.

Hence, by means of the endoscope apparatus of the modification of the present embodiment also, each observation image obtained by an endoscope that is capable of observing in three directions can be made an appropriate brightness.

### Fourth Embodiment

An endoscope system of the present embodiment is an endoscope system that can reduce illuminating light in only a region in which halation occurred in an endoscopic image.

FIG. 28 is a configuration diagram that illustrates the configuration of an endoscope system relating to the present embodiment. An endoscope system 1C of the present embodiment has substantially the same configuration as the endoscope system 1 of the first embodiment, and hence components that are the same as in the endoscope system 1 are denoted by the same reference numerals and a description of such components is omitted below, and components that are different from those of the endoscope system 1 are described.

As shown in FIG. 28, the illuminating window 7 and the observation window 8 for front observation and three illuminating windows 9 for upward, leftward and rightward observation are provided in the distal end portion 6a of the insertion portion 6. The distal end portion 34b of a light guide 34C branches into four branches, and the respective branch ends are arranged at the rear of the respective illuminating windows 7 and 9 as illuminating light emitting portions.

A light adjustment portion 31C includes the polarization filter 111, the polarization filter 112 and the diaphragm 31c.

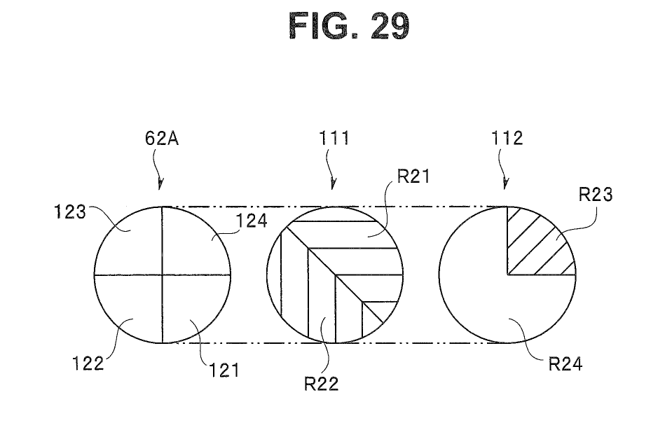

FIG. 29 is a view that illustrates the configuration of the polarization filters 111 and 112 as a light amount adjustment portion, and also illustrates the correspondence relation between four regions 121, 122, 123 and 124 of the proximal end portion of the light guide 34C and respective areas R21, R22, R23 and R24 of the two polarization filters 111 and 112.

The plurality of polarization filters as an example of a light amount adjustment portion have, for example, respective portions that are disposed substantially collinearly so as to lie along the optical axis of the illuminating light.

The proximal end portion 34a of the light guide 34C is divided into four regions so that the areas are equal around the central axis of the light guide 34C. In FIG. 29, a right-lower region 121 is the region of an end portion of a light guide for front illumination, a left-lower region 122 is the region of an end portion of a light guide for left side illumination, a left-upper region 123 is the region of an end portion of a light guide for upward illumination, and a right-upper region 124 is the region of an end portion of a light guide for right side illumination.

That is, at the proximal end portion 34a of the light guide 34C, the region 124 for right side illumination and the region 122 for left side illumination are disposed on opposing sides on the incident surface 62A, and the region 123 for upward illumination and the region 121 for front illumination are disposed on opposing sides on the incident surface 62A.

The circular polarization filter 111 is divided into two parts by a straight line that passes through the center of the circular polarization filter 111, and has two semicircular areas R21 and R22. The areas R21 and R22 have slits that are provided in directions that are orthogonal to each other.

The circular polarization filter 112 is divided into four parts by lines that pass through the center of the circular polarization filter 112, and slits having the same width as the slits of the polarization filter 111 are provided in one quadrantal area R23 among the four parts. Slits are not formed in an area R24 that is other than the area R23 of the polarization filter 112. The shape and size of the area R23 matches the shape and size of the each of the four regions 121, 122, 123 and 124.

As shown in FIG. 29, the polarization filter 111 is disposed and fixed with respect to the four regions from 121 to 124 of the incident surface 62A of the light guide 34C so that light of equal amounts that passes through the areas R21 and R22 of the polarization filter 111 is incident on the first region 121 and the third region 123, light that passes through the area R22 of the polarization filter 111 is incident on the second region 122, and light that passes through the area R21 of the polarization filter 111 is incident on the fourth region 124.

The polarization filter 112 is rotatable with respect to the polarization filter 111.

In this case, a rotational angle θ7 of the polarization filter 112 with respect to the polarization filter 111 when the area R23 of the polarization filter 112 matches the region 124 of the incident surface 62A of the light guide 34C, that is, when light from the area R23 passes through the polarization filter 111 and is incident on only the region 124, is taken as 0 degrees.

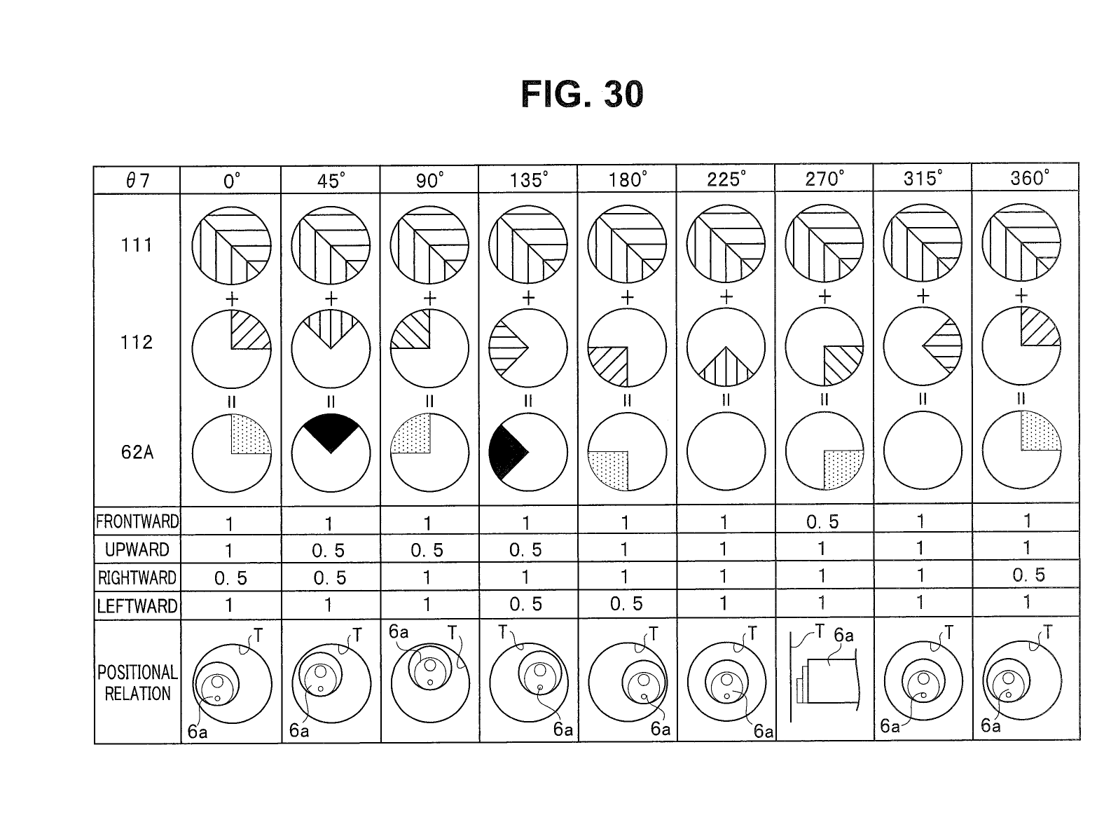

FIG. 30 is a chart for describing the rotation angle θ7 and light amounts incident on the incident surface 62A of the light guide 34C.

FIG. 30 shows that, when the polarization filter 112 rotates, a light amount that is incident on the incident surface 62A of the light guide 34C changes in accordance with the angle θ7.

For example, it is shown that when the rotational angle θ7 of the polarization filter 112 with respect to the polarization filter 111 is 0 degrees, although 100% of light is incident on the regions 121, 122 and 123, 50% of light is incident on the region 124 for right side illumination. In FIG. 30, the fact that only half of the light is incident on the region 124 for right side illumination is indicated by a region with dot hatching.

Further, it is shown that when the rotational angle θ7 is 45 degrees, although 100% of light is incident on the regions 121 and 122, 50% of light is incident on the region 123 for upward illumination and the region 124 for right side illumination. In FIG. 30, a fact that no light is incident on one half of each of the region 123 for upward illumination and the region 124 for right side illumination is indicated by a black region.

Likewise, FIG. 30 shows how much light is incident on the respective regions of the incident surface 62A of the light guide 34C at respective angles when the rotation angle θ7 changes from 0 degrees up to 360 degrees. On the incident surface 62A of the light guide 34C, a region with dot hatching indicates a region on which 50% of light is incident, and a black region indicates a region on which 0% of light is incident.

A middle row section in FIG. 30 shows illumination light amounts for each of front illumination, upward illumination, rightward illumination and leftward illumination. In addition, a lower row section in FIG. 30 shows positions of an inner wall T of a subject and the distal end portion 6a of the insertion portion 6.

When the insertion portion 6 is inserted into a subject and the distal end portion 6a of the insertion portion 6 is close to an inner wall inside the subject, a region in a direction that is too close to the inner wall appears as a halation region in the endoscopic image.

For example, when the right side of the distal end portion 6a is too close to an inner wall, a region on the right side of the endoscopic image appears as a halation region. Further, when an upper part of the distal end portion 6a is too close to an inner wall, a region on an upper side of the endoscopic image appears as a halation region. In addition, when the front of the distal end portion 6a is too close to an inner wall, a region on the front side of the endoscopic image appears as a halation region.

The control portion 42 can determine which region of an endoscopic image halation occurs in based on the luminance value of each pixel in the respective regions of the endoscopic image.

Therefore, when the control portion 42 detects a halation region, the control portion 42 rotates the polarization filter 112 so as to decrease the amount of illuminating light which illuminates the relevant region. As a result, a halation region can be eliminated from the endoscopic image. That is, the drive portion 32 controls the drive portion 32 so as to drive the light adjustment portion 31C so that halation as a photometry result from the photometry portion 41 is reduced.

When the angle θ7 in FIG. 30 is 225 degrees or 315 degrees, as shown in the lower row, the distal end portion 6a of the insertion portion 6 is not too close to the inner wall T inside the subject. Consequently, light that is incident on the incident surface 62A of the light guide 34C is evenly incident thereon.

For example, when the distal end portion 6a of the insertion portion 6 is too close to the inner wall T on the right side inside the subject, a region on the right side of the endoscopic image appears as a halation region. The control portion 42 can detect that the region in which halation is occurring is the region on the right side based on the brightness of each region of the endoscopic image. In such a case, in FIG. 30, as shown in the lower row for a time when the angle θ7 is 0 degrees, the right side of the distal end portion 6a is too close to the inner wall T.

However, when the control portion 42 controls the drive portion 32 so as to make the angle θ7 of the polarization filter 112 with respect to the polarization filter 111 0 degrees, as shown in the middle row for a time when the angle θ7 is 0 degrees in FIG. 30, only the light for right side illumination can be reduced to a half.

Further, similarly, for example, when the distal end portion 6a of the insertion portion 6 is too close to the inner wall T on the upper side inside the subject, although the region on the upper side of the endoscopic image becomes a halation region, in this case, when the control portion 42 controls the drive portion 32 to make the angle θ7 of the polarization filter 112 with respect to the polarization filter 111 90 degrees, in FIG. 30, as shown in the middle row for a time when the angle θ7 is 90 degrees, only light for upward illumination can be reduced to a half.

Similarly, when halation is detected in other regions including also the front region, the halation region in the endoscopic image can be eliminated or the halation can be suppressed to a certain extent by controlling the rotational angle θ7 of the polarization filter 112.

As described above, according to the endoscope system of the present embodiment, the illuminating light of only a region in which halation has occurred in an endoscopic image can be decreased to thereby eliminate or suppress the occurrence of halation in the endoscopic image.

### Fifth Embodiment

An endoscope system of the present embodiment is an endoscope system that can adjust light amounts of front illumination and illumination in three lateral directions.

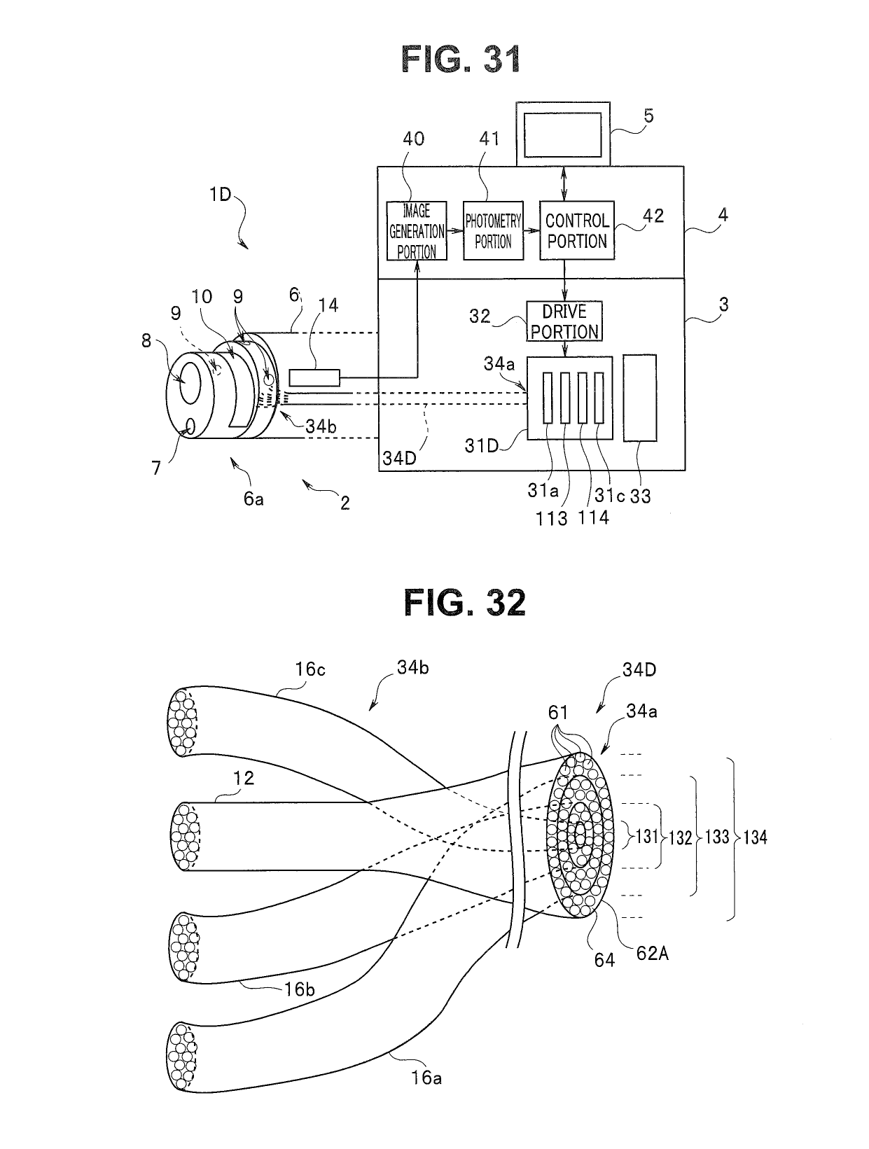

FIG. 31 is a configuration diagram that illustrates the configuration of an endoscope system relating to the present embodiment. An endoscope system 1D of the present embodiment has substantially the same configuration as the endoscope systems 1 and 1C of the first and fourth embodiments, and hence components that are the same as in the endoscope systems 1 and 1C are denoted by the same reference numerals and a description of such components is omitted below, and components that are different from those of the endoscope systems 1 and 1C are described.

As shown in FIG. 31, the illuminating window 7 and the observation window 8 for front observation and the three illuminating windows 9 for upward, leftward and rightward observation are provided in the distal end portion 6a of the insertion portion 6, and the distal end portion 34b of a light guide 34D branches into four branches.

The light adjustment portion 31D includes the polarization filter 31a and the two polarization filters 113 and 114 as a light amount adjustment portion and the diaphragm 31c.

FIG. 32 is a schematic view that illustrates the configuration of the light guide 34D relating to the present embodiment. The light guide 34D is constituted by bundling the large number of optical fibers 61. A proximal end portion 34a of the light guide 34D has a circular incident surface 62A on which light that passed through the light adjustment portion 31D is incident. End faces of the large number of optical fibers 61 are gathered together at the incident surface 62A.

The plurality of polarization filters as an example of a light amount adjustment portion have, for example, respective portions that are disposed substantially collinearly so as to lie along the optical axis of the illuminating light.

The incident surface 62A has four regions 131, 132, 133 and 134 that are light-receiving regions. An optical fiber group having end portions in the first region 131 is a third light guide for lateral illumination 16c. An optical fiber group having end portions in the second region 132 is the second light guide for lateral illumination 16b. An optical fiber group having end portions in the third region 133 is the first light guide for lateral illumination 16a. An optical fiber group having end portions in the fourth region 134 is the light guide for front illumination 12.

In FIG. 32, the proximal end portions of the light guide for front illumination 12 are held together and disposed in the circular ring-shaped fourth region 134 on the outermost circumferential side of the circular incident surface 62A of the proximal end portion 34a of the light guide 34D. The proximal end portions of the first light guide for lateral illumination 16a are held together and disposed in the circular ring-shaped third region 133 on the inner side of the fourth region 134. The proximal end portions of the second light guide for lateral illumination 16b are held together and disposed in the circular ring-shaped second region 132 on the inner side of the third region 133. The proximal end portions of the third light guide for lateral illumination 16c are held together and disposed in the circular region 131 on the inner side of the second region 132.

Note that a partition film may be provided between the light guide for front illumination 12 and the light guide for lateral illumination 16a, between the light guide for lateral illumination 16a and the light guide for lateral illumination 16b, and between the light guide for lateral illumination 16b and the light guide for lateral illumination 16c so that light does not leak between the aforementioned light guides.

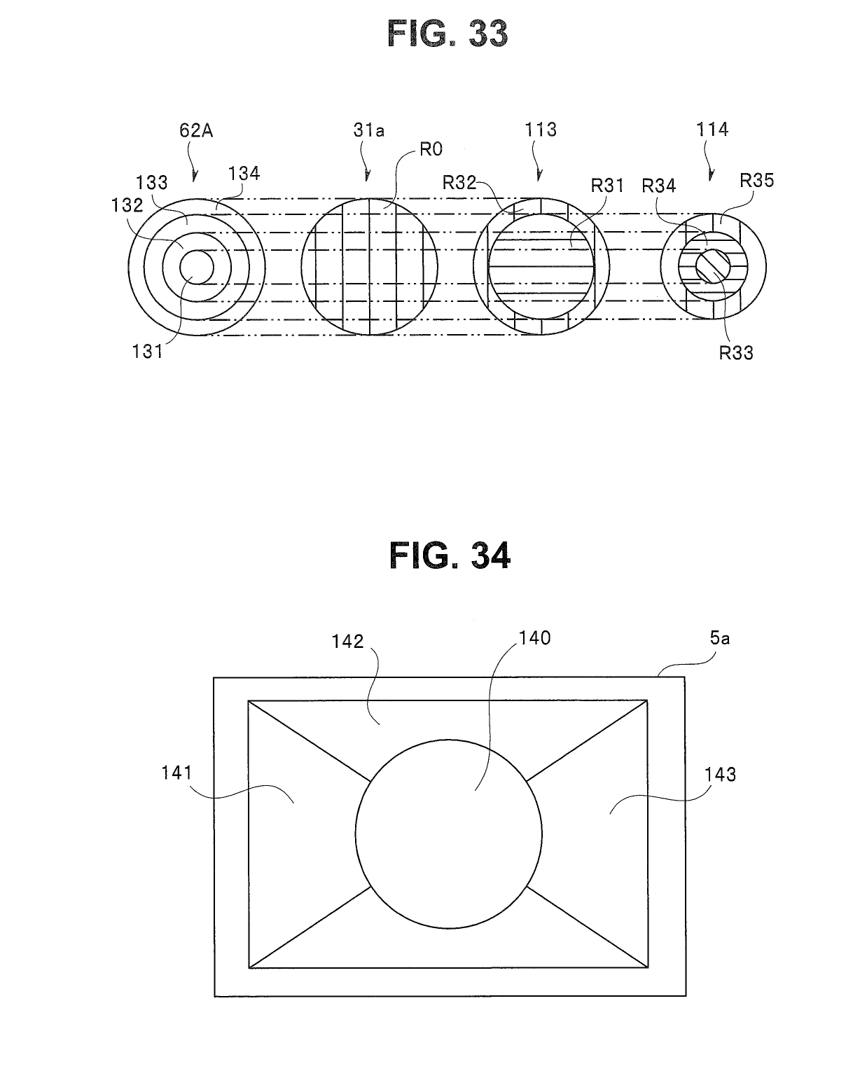

FIG. 33 is a view illustrating the correspondence relation between the four regions 131, 132, 133 and 134 of the incident surface 62A of the light guide 34D and the respective areas R31, R32, R33, R34 and R35 of the three polarization filters 31a, 121 and 122.

The polarization filter 113 has a central circular area R31 and a circular ring-shaped area R32 around the area R31. Slits having the same width as the slits of the polarization filter 31a are provided in the areas R31 and R32. The direction of the slits in the area R31 and the direction of the slits in the area R32 are orthogonal.

The polarization filter 114 has a central circular area R33, a circular ring-shaped area R34 provided around the area R33, and a circular ring-shaped area R35 provided around the area R34. Slits having the same width as the slits of the polarization filter 31a are provided in the areas R33, R34 and R35. The direction of the slits in the area R34 and the direction of the slits in the area R35 are orthogonal. The direction of the slits in the area R33 is at an angle of 45 degrees with respect to the direction of the slits in the areas R34 and R35.

As shown in FIG. 33, the outer diameter of the polarization filter 31a, the outer diameter of the polarization filter 113, and the outer diameter of the region 134 of the incident surface 62A of the light guide 34D are equal. The outer diameter of the area R31 of the polarization filter 113 and the outer diameter of the region 133 of the incident surface 62A are equal. In addition, the outer diameter of the area R31 of the polarization filter 113 and the outer diameter of the area R35 of the polarization filter 114 are equal.

Further, the outer diameter of the area R34 of the polarization filter 114 and the outer diameter of the region 132 of the incident surface 62A are equal. Furthermore, the outer diameter of the area R33 of the polarization filter 114 and the outer diameter of the region 131 of the incident surface 62A are equal.

The respective polarization filters 31a, 121 and 122 are disposed with respect to the incident surface 62A of the light guide 34D so that light emitted from the area R32 of the polarization filter 113 is transmitted through the polarization filter 31a and is incident on the fourth region 134 of the incident surface 62A.

The respective polarization filters 31a, 121 and 122 are disposed with respect to the incident surface 62A of the light guide 34D so that light emitted from the area R35 of the polarization filter 114 is transmitted through the area R31 of the polarization filter 113 and, furthermore, is transmitted through the polarization filter 31a to be incident on the third region 133 of the incident surface 62A.

The respective polarization filters 31a, 121 and 122 are disposed with respect to the incident surface 62A of the light guide 34D so that light emitted from the area R34 of the polarization filter 114 is transmitted through the area R31 of the polarization filter 113 and, furthermore, is transmitted through the polarization filter 31a to be incident on the second region 132 of the incident surface 62A.

The respective polarization filters 31a, 121 and 122 are disposed with respect to the incident surface 62A of the light guide 34D so that light emitted from the area R33 of the polarization filter 114 is transmitted through the area R31 of the polarization filter 113 and, furthermore, is transmitted through the polarization filter 31a to be incident on the first region 131 of the incident surface 62A.

By rotating the polarization filter 113 within a range of 0 degrees to 90 degrees relative to the polarization filter 31a, the amount of illumination light for a front observation image and the three amounts of illumination light for a lateral observation image can be balanced.

In addition, after the illumination for a front observation image and the three amounts of illumination light for a lateral observation image are balanced, the illumination for a first lateral observation image, the illumination for a second lateral observation image, and the illumination for a third lateral observation image can be balanced by rotating the polarization filter 114 within a range from −90 degrees to 90 degrees relative to the polarization filter 113.

FIG. 34 is a view illustrating an example of a display screen of an endoscopic image that is displayed on the display apparatus 5.

Four endoscopic images are displayed on the display screen 5a of the display apparatus 5. A circular first region 140 in the center is a region that displays a front observation image which is generated based on an image pickup signal from the image pickup unit 14c. A second region 141 that is to the left of the first region 140 is a region that displays a first lateral observation image which is generated based on an image pickup signal from the image pickup unit 14a. A third region 142 on the upper side of the first region 140 is a region that displays a second lateral observation image which is generated based on an image pickup signal from the image pickup unit 14a. A fourth region 143 that is to the right of the first region 140 is a region that displays a third lateral observation image which is generated based on an image pickup signal from the image pickup unit 14a.

As shown in FIG. 34, the four endoscopic images are displayed on the display screen 5a of the display apparatus 5. The photometry portion 41 of the processor 4 calculates the brightness of each region of the endoscopic images and outputs the calculated values to the control portion 42.

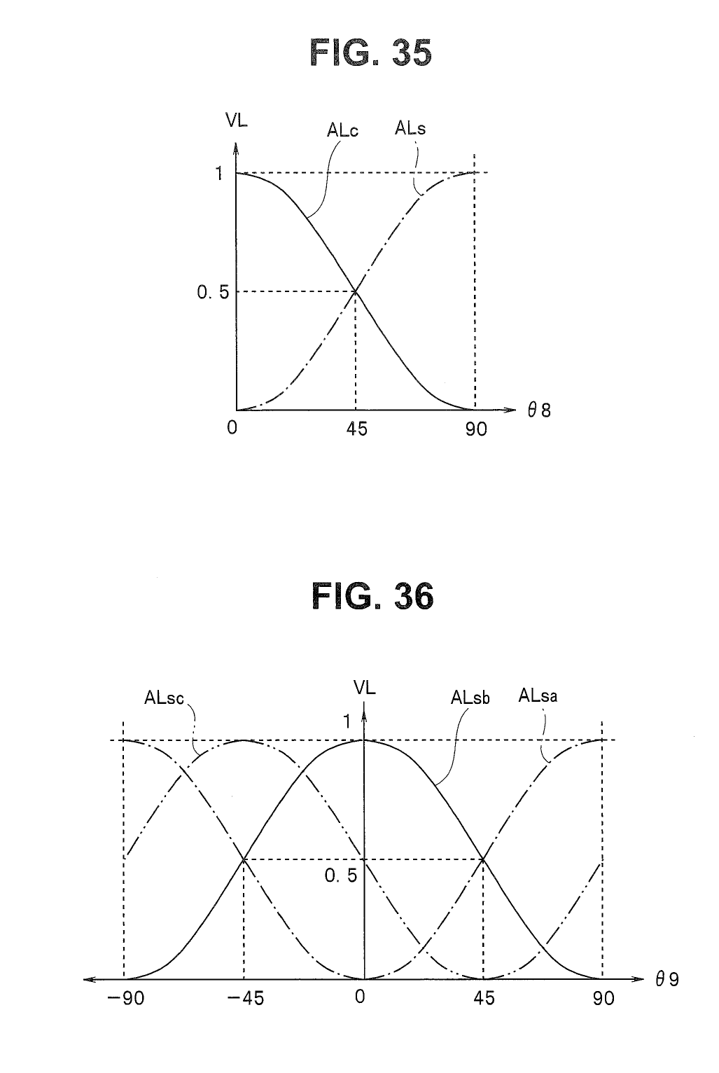

FIG. 35 is a graph illustrating the relation between a rotational angle θ8 of the polarization filter 113 with respect to the polarization filter 31a, a light amount VL that is incident on the region 134 of the light guide 34D, and a light amount VL that is incident on the three regions 131, 132 and 133.

In this case, the rotational angle θ8 of the polarization filter 113 with respect to the polarization filter 31a when the direction of the slits of the polarization filter 31a and the direction of the slits of the area R32 of the polarization filter 113 are parallel is taken as 0 degrees.

When the rotational angle θ8 of the polarization filter 113 with respect to the polarization filter 31a changes from 0 degrees toward 90 degrees, the light amount VL incident on the region 134 of the light guide 34D gradually decreases as shown by a solid line ALc, and the light amount VL incident on the regions 131, 132 and 133 of the light guide 34D gradually increases as shown by an alternate long and short dashed line ALs.

When the rotational angle θ8 of the polarization filter 113 is 0 degrees, as shown in FIG. 35, the light amount VL that is incident on the region 134 is 1 (that is, 100% transmission) and the light amount VL incident on the regions 131, 132 and 133 is 0 (that is, 0% transmission). When the rotational angle θ8 of the polarization filter 113 is 90 degrees, as shown in FIG. 35, the light amount VL that is incident on the region 134 is 0 (that is, 0% transmission) and the light amount VL incident on the regions 131, 132 and 133 is 1 (that is, 100% transmission).

When the rotational angle θ8 of the polarization filter 113 is 45 degrees, the light amount VL that is incident on the region 134 and the light amount VL that is incident on the regions 131, 132 and 133 are each 0.5 (that is, 50% transmission).

Thus, by changing the rotational angle θ8 of the polarization filter 113 within the range from 0 to 90 degrees, the distribution between the light amount incident on the region 134 and the light amount incident on the three regions 131, 132 and 133 can be changed.

FIG. 36 is a graph illustrating the relation between a rotational angle θ9 of the polarization filter 114 with respect to the polarization filter 113 and a light amount incident on each of the regions 131, 132 and 133 of the light guide 34D.

In this case, the rotational angle θ9 of the polarization filter 114 with respect to the polarization filter 113 when the direction of the slits of the area R32 of the polarization filter 113 and the direction of the slits of the area R35 of the polarization filter 114 are parallel is taken as 0 degrees.

When the rotational angle θ9 of the polarization filter 114 with respect to the polarization filter 113 changes from 0 degrees toward 90 degrees, and when the rotational angle θ9 changes from 0 degrees toward −90 degrees, the light amount VL incident on the region 133 of the light guide 34D gradually increases as shown by an alternate long and short dashed line ALsa, and the light amount VL incident on the region 132 of the light guide 34D gradually decreases as shown by a solid line ALsb.

Further, when the rotational angle θ9 of the polarization filter 114 with respect to the polarization filter 113 changes from 0 degrees toward 90 degrees, the light amount VL that is incident on the region 131 of the light guide 34D gradually decreases and thereafter increases as shown by a chain double-dashed line ALsc. When the rotational angle θ9 of the polarization filter 114 with respect to the polarization filter 113 changes from 0 degrees toward −90 degrees, the light amount VL that is incident on the region 131 of the light guide 34D gradually decreases and thereafter increases as shown by a chain double-dashed line ALsc.

When the rotational angle θ9 of the polarization filter 114 with respect to the polarization filter 113 is 45 degrees or −45 degrees, as shown in FIG. 36, the light amount VL incident on the regions 132 and 133 becomes 50%.

Thus, by changing the rotational angle θ9 of the polarization filter 114 within a range from −90 degrees to 90 degrees, the distribution of a light amount incident on the regions 131, 132 and 133 of the light guide 34D can be changed.

Next, operations of the processor 4 are described.

As described above, the photometry portion 41 calculates the brightness of the respective images of the regions 140, 141, 142 and 143 in an endoscopic image, and outputs the calculated brightness values to the control portion 42. The brightness of each region is an average value of the luminance of all pixels within the relevant region. The control portion 42 drives the drive portion 32 to rotate the polarization filter 113 so that a brightness La1 of an image of the region 140 that displays a front observation image and the brightness Las of images of the other three regions 141, 142 and 143 become equal.

Rotational control of the polarization filter 113 is performed by feedback control that, for example, while monitoring the brightness La1 and brightness Las, drives the drive portion 32 within a range in which the rotational angle θ8 is from 45 degrees to 90 degrees when the brightness La1 is greater than the brightness Las, and drives the drive portion 32 within a range in which the rotational angle θ8 is from 0 degrees to 45 degrees when the brightness La1 is less than the brightness Las, so that the brightness La1 and brightness Las become equal.

In addition, after the brightness La1 and brightness Las become equal, the control portion 42 drives the drive portion 32 to rotate the polarization filter 114 so that the brightnesses of the images of the three regions 141, 142 and 143 become equal.

Rotational control of the polarization filter 114 is performed by feedback control that, for example, while monitoring the brightnesses of the three regions 141, 142 and 143, drives the drive portion 32 within a range in which the rotational angle θ9 is from −90 degrees to 90 degrees so that the brightnesses of the three regions 141, 142 and 143 become equal.

As described above, according to the endoscope apparatus of the present embodiment, each observation image obtained by an endoscope that is capable of observing in four directions can be made an appropriate brightness.

### Sixth Embodiment

An endoscope system of the present embodiment is an endoscope system that can adjust illumination light amounts for four directions.

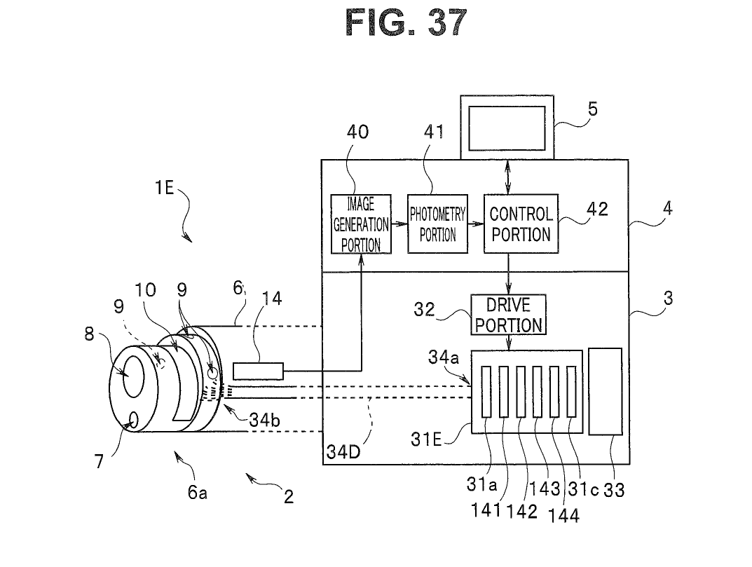

FIG. 37 is a configuration diagram that illustrates the configuration of the endoscope system relating to the present embodiment. An endoscope system 1E of the present embodiment has substantially the same configuration as the endoscope system 1D of the fifth embodiment, and hence components that are the same as in the endoscope system 1D are denoted by the same reference numerals and a description of such components is omitted below, and components that are different from those of the endoscope system 1D are described.

As shown in FIG. 37, the illuminating window 7 and the observation window 8 for front observation and three illuminating windows 9 for upward, leftward and rightward observation are provided in the distal end portion 6a of the insertion portion 6, and the distal end portion 34b of the light guide 34D branches into four branches.

A light adjustment portion 31E includes the polarization filter 31a and four polarization filters 141, 142, 143 and 144, and the diaphragm 31c.

The plurality of polarization filters as an example of a light amount adjustment portion have, for example, respective portions that are disposed substantially collinearly so as to lie along the optical axis of the illuminating light.

The drive portion 32 can individually rotate each of the four polarization filters 141, 142, 143 and 144 as a light amount adjustment portion independently from each other.

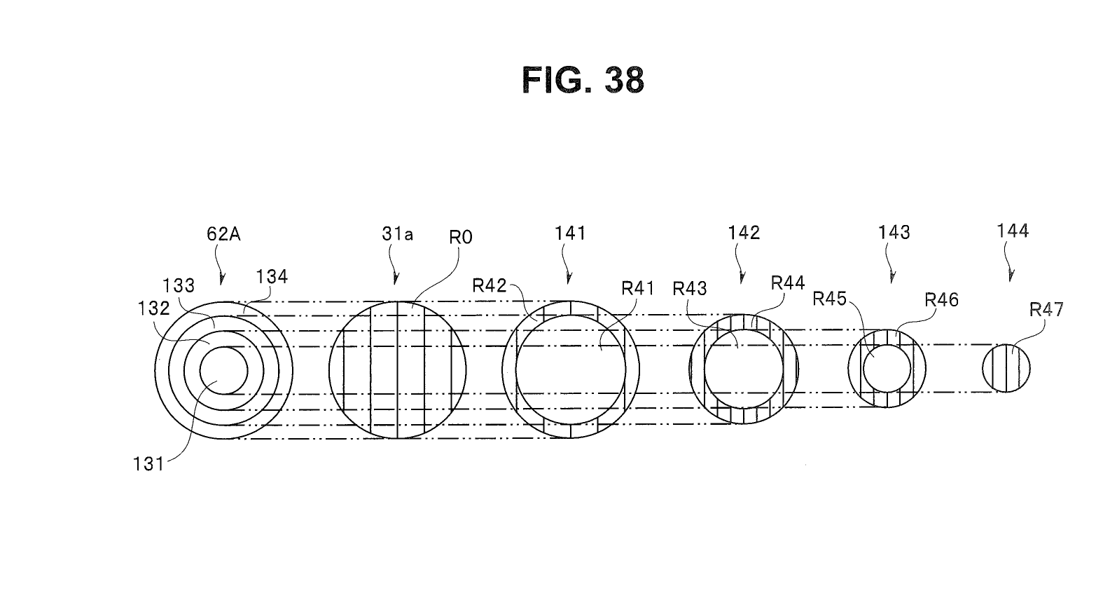

FIG. 38 is a view illustrating the correspondence relation between the four regions 131, 132, 133 and 134 of the proximal end portion of the light guide 34 and respective areas R41, R42, R43, R44, R45 and R46 of the four polarization filters 141, 142, 143 and 144.

The polarization filter 141 has a central circular area R41, and a circular ring-shaped area R42 around the area R41. Slits having the same width as the slits of the polarization filter 31a are provided in the area R42. The area R41 does not have slits, and is a region that transmits incident light as it is. That is, only the area R42 is a polarization filter region.

The polarization filter 142 has a central circular area R43, and a circular ring-shaped area R44 around the area R43. Slits having the same width as the slits of the polarization filter 31a are provided in the area R44. The area R43 does not have slits, and is a region that transmits incident light as it is. That is, only the area R44 is a polarization filter region.

The polarization filter 143 has a central circular area R45, and a circular ring-shaped area R46 around the area R45. Slits having the same width as the slits of the polarization filter 31a are provided in the area R46. The area R45 does not have slits, and is a region that transmits incident light as it is. That is, only the area R46 is a polarization filter region.

The polarization filter 144 has a circular area R47 in which slits having the same width as the slits of the polarization filter 31a are provided.

As shown in FIG. 38, the outer diameter of the polarization filter 31a, the outer diameter of the polarization filter 141 and the outer diameter of the region 134 of the incident surface 62A of the light guide 34D are equal. The outer diameter of the area R44 of the polarization filter 142, the inner diameter of the area R42 of the polarization filter 141 and the outer diameter of the region 133 of the incident surface 62A are equal.

In addition, the outer diameter of the area R46 of the polarization filter 143, the inner diameter of the area R44 of the polarization filter 142 and the outer diameter of the region 132 of the incident surface 62A are equal.

Further, the outer diameter of the area R47 of the polarization filter 144, the inner diameter of the area R46 of the polarization filter 143 and the outer diameter of the region 131 of the incident surface 62A are equal.

The respective polarization filters 31a and 141 are disposed with respect to the incident surface 62A of the light guide 34D so that light emitted from the area R42 of the polarization filter 141 passes through the polarization filter 31a and is incident on the fourth region 134 of the incident surface 62A.

The respective polarization filters 31a, 141 and 142 are disposed with respect to the incident surface 62A of the light guide 34D so that light emitted from the area R44 of the polarization filter 142 passes through the area R41 of the polarization filter 141 and also passes through the polarization filter 31a and is incident on the third region 133 of the incident surface 62A.

The respective polarization filters 31a, 141, 142 and 143 are disposed with respect to the incident surface 62A of the light guide 34D so that light emitted from the area R46 of the polarization filter 143 passes through the area R43 of the polarization filter 142, passes through the area R41 of the polarization filter 141 and, further, passes through the polarization filter 31a and is incident on the second region 132 of the incident surface 62A.

The respective polarization filters 31a, 141, 142, 143 and 144 are disposed with respect to the incident surface 62A of the light guide 34D so that light emitted from the area R47 of the polarization filter 144 passes through the area R45 of the polarization filter 143, passes through the area R43 of the polarization filter 142, passes through the area R41 of the polarization filter 141 and, further, passes through the polarization filter 31a and is incident on the first region 131 of the incident surface 62A.

By the respective polarization filters 141, 142, 143 and 144 being independently rotated within a range from 0 degrees to 90 degrees relative to the polarization filter 31a, the light amount of the illumination for a front observation image and the three light amounts for illumination for a lateral observation image can be balanced.

The control portion 42 independently drives the respective polarization filters 141, 142, 143 and 144 so that the brightnesses of the respective regions in the endoscopic image become equal.

As described above, according to the endoscope apparatus of the present embodiment, light amounts for illumination in four directions can be independently adjusted, and observation images for four directions can be made the appropriate brightness.

As described in the foregoing, according to the respective embodiments and respective modifications described above, an endoscope system can be provided in which respective observation images obtained by an endoscope that is capable of observation in two or more directions can be made the appropriate brightness.

The present invention is not limited to the above embodiments and various changes and modifications can be made within a range that does not depart from the spirit and scope of the present invention.

What is claimed is:

1. An endoscope system, comprising:
an insertion portion to be inserted inside a subject;
an illuminating light emitting portion configured to emit a first illuminating light toward a front region inside the subject which includes a front direction of the insertion portion that is approximately parallel to a longitudinal direction of the insertion portion, and to emit a second illuminating light toward a lateral region in which at least one part is different from the front region inside the subject and which includes a lateral direction of the insertion portion that intersects with the longitudinal direction of the insertion portion;
a first subject image acquisition portion which is provided in the insertion portion and is configured to acquire a first subject image from the front region;
a second subject image acquisition portion which is provided in the insertion portion and is configured to acquire a second subject image from the lateral region;
an image generation portion configured to generate a front observation image based on the first subject image and generate a lateral observation image based on the second subject image;
a brightness comparison portion configured to compare a brightness of the front observation image and a brightness of the lateral observation image;
a light amount adjustment portion configured to adjust an amount of the second illuminating light; and
a drive portion configured to drive the light amount adjustment portion so that the lateral observation image becomes approximately a same brightness as the front observation image, based on a result of comparing the brightnesses obtained by the brightness comparison portion.

2. The endoscope system according to claim 1, wherein:
the light amount adjustment portion comprises a first polarization filter, and a second polarization filter having first and second areas, two polarization directions of which are orthogonal to each other, in which one of the first polarization filter and the second polarization filter is rotatable.

3. The endoscope system according to claim 2, wherein:
the second polarization filter is either one of:
a polarization filter having the first area and the second area which is provided around the first area, and
a polarization filter having a third area including two areas whose polarization directions are orthogonal to each other at a center part, the first area that is provided around the third area, and the second area that is provided around the first area.

4. The endoscope system according to claim 1, further comprising:
a light-receiving portion configured to receive an illuminating light for illuminating inside of the subject which is supplied from a light source, at each of a plurality of light-receiving regions in a cross-sectional direction; and
a light-guiding portion configured to guide light into the insertion portion and emit the illuminating light that is received by the light-receiving portion as the first and the second illuminating light toward the front and the lateral regions, respectively.

5. The endoscope system according to claim 4, wherein:
the light-receiving portion is configured to receive the illuminating light at three light-receiving regions, and
the light-guiding portion is configured to emit the illuminating light that is received at a first light-receiving region of the three light-receiving regions toward the front region as the first illuminating light, emit the illuminating light that is received at a second light-receiving region of the three light-receiving regions toward the lateral region as the second illuminating light, and emit the illuminating light that is received at a third light-receiving region of the three light-receiving regions toward a third region as a third illuminating light.

6. The endoscope system according to claim 1, wherein the light amount adjustment portion comprises a first polarization filter that is fixed, a second polarization filter that is rotatable with respect to the first polarization filter, and a third polarization filter that is rotatable with respect to the second polarization filter.

7. The endoscope system according to claim 6, wherein:
the second polarization filter has a first area that is provided at a center part, and a second area that is provided around the first area; and
the third polarization filter has a third area that is provided at a center part, and a fourth area that is provided around the third area.

8. The endoscope system according to claim 7, wherein the light amount adjustment portion is either one of:
a configuration in which the second area and the fourth area have a polarization filter region; and
a configuration in which the third area has a polarization direction of 45 degrees relative to a polarization direction of a fifth area that is provided around a polarization direction of the fourth area.

9. The endoscope system according to claim 4, wherein:
the light-receiving portion receives the illuminating light at each of four regions;
the light-guiding portion emits the illuminating light that is received at each of the four regions toward four regions; and
the light amount adjustment portion comprises a fixed first polarization filter having first and second areas, two polarization directions of which are orthogonal to each other, and a second polarization filter which has areas that match a shape and a size of the respective regions and which is rotatable with respect to the first polarization filter.

10. The endoscope system according to claim 1, wherein:
the light amount adjustment portion has a fixed first polarization filter, and a second polarization filter, a third polarization filter, a fourth polarization filter and a fifth polarization filter that are rotatable with respect to the first polarization filter;
the second polarization filter has a first area at a center part, and has a second area that is provided around the first area;
the third polarization filter has a third area at a center part, and has a fourth area that is provided around the third area;
the fourth polarization filter has a fifth area at a center part, and has a sixth area that is provided around the fifth area;
the fifth polarization filter has a seventh area; and
the second area, the fourth area, the sixth area and the seventh area include a filter configured to adjust a light amount.

11. The endoscope system according to claim 1, further comprising:
a display portion into which a first image signal that is based on the front observation image and a second image signal that is based on the lateral observation image are inputted from the image generation portion, and which is configured to display an endoscopic image in which the lateral observation image is arranged next to the front observation image.

12. The endoscope system according to claim 11, wherein:
the front observation image is displayed on the display portion so as to be an approximately circular shape, and
the lateral observation image is displayed on the display portion so as to be an annular shape that surrounds at least one part of a circumference of the front observation image.

13. The endoscope system according to claim 1, wherein:
the first subject image acquisition portion is disposed facing a direction in which the insertion portion is inserted, in a distal end portion of the insertion portion; and
the second subject image acquisition portion is disposed facing a radial direction of the insertion portion, in a side face portion of the insertion portion;
the endoscope system further comprising:
a first image pickup portion which is configured to photoelectrically convert the first subject image from the first subject image acquisition portion and is electrically connected to the image generation portion; and
a second image pickup portion that is different to the first image pickup portion, and which is configured to photoelectrically convert the second subject image from the second subject image acquisition portion and is electrically connected to the image generation portion.

14. The endoscope system according to claim 1, wherein:
the first subject image acquisition portion is disposed facing a direction in which the insertion portion is inserted, in a distal end portion of the insertion portion; and
the second subject image acquisition portion is disposed facing a radial direction of the insertion portion, in a side face portion of the insertion portion;
the endoscope system further comprising:
an image pickup portion which is disposed so as to photoelectrically convert, at a same image pickup surface, the first subject image from the first subject image acquisition portion and the second subject image from the second subject image acquisition portion, and which is electrically connected to the image generation portion.

15. The endoscope system according to claim 1, further comprising:
a photometry portion configured to measure a brightness in a predetermined region of the front observation image and the lateral observation image that the image generation portion generates, wherein:
the drive portion drives the light amount adjustment portion so that a brightness of the front observation image and a brightness of the lateral observation image as photometry results from the photometry portion become approximately a same brightness, or so that halation of the lateral observation image is reduced.
